(19) United States
(12) Patent Application Publication
(10) Pub. No.: US 2003/0125251 A1
(43) Pub. Date: Jul. 3, 2003

(54) Transforming growth factor beta (TGF-beta) antagonist selectively neutralizes "pathological" TGF-beta

(76) Inventors: Lalage M. Wakefield, Chevy Chase, MD (US); Yu-an Yang, Baltimore, MD (US)

Correspondence Address: KLARQUIST SPARKMAN, LLP, One World Trade Center, Suite 1600, 121 SW Salmon Street, Portland, OR, 97204-2988, US

(21) Appl. No.: 10/176,266

(22) Filed: Jun. 20, 2002

Related U.S. Application Data
(60) Provisional application No. 60/300,087, filed on Jun. 21, 2001.

(51) Int. Cl. A61K 39/395; A61K 38/17

(52) U.S. Cl. 514/12; 424/155.1

(57) ABSTRACT

The present invention provides methods and compositions for the suppression of metastasis by a soluble TGF-β antagonist (SR2F). This antagonist is composed of the soluble extracellular domain of the type II TGF-β receptor fused to the Fc domain of human IgG. In particular, the present invention is directed to the use of SR2F to prevent metastasis without affecting the normal physiological role of TGF-β. Thus, the SR2F of the present invention discriminates between “physiological” TGF-β and “pathological” TGF-β in such a manner that only the “pathological” TGF-β is affected by the administration of SR2F.

This application claims priority to Provisional Application Ser. No. 60/300,087 filed on Jun. 21, 2001, which is herein incorporated by reference in its entirety. This invention was made in part during work partially supported by Federal funds from the National Cancer Institute, the National Institutes of Health under contract no. NO1-CO-12400.

### FIELD OF THE INVENTION

The present invention provides methods and compositions for the suppression of metastasis by a soluble TGF-β antagonist (SR2F). This antagonist is composed of the soluble extracellular domain of the type II TGF-β receptor fused to the Fc domain of human IgG. In particular, the present invention is directed to the use of SR2F to prevent metastasis without affecting the normal physiological role of TGF-β. Thus, the SR2F of the present invention discriminates between “physiological” TGF-β and “pathological” TGF-β in such a manner that only the “pathological” TGF-β is affected by the administration of SR2F.

### BACKGROUND OF THE INVENTION

Transforming growth factor beta (TGF-β) is a member of a large superfamily of growth factors (cytokines) involved in the regulation of various biological processes, including cell proliferation and differentiation, extracellular matrix metabolism, bone morphogenesis, adhesion, apoptosis, cell migration, embryogenesis, tissue repair, and immune system modulation. Virtually every cell in the body (e.g., epithelial, endothelial, epithelial, hematopoietic, neuronal, and connective tissue cells) produces and has receptors for TGF-β.

Increases and decreases in TGF-β have been associated with numerous diseases, including atherosclerosis and fibrotic diseases of the kidney, liver, and lung. Genetic mutations in TGF-β, its receptors, and/or intracellular signaling molecules associated with TGF-β are also important in pathogenic processes, particularly in cancer and hereditary hemorrhagic telangiectasia.

There are multiple isoforms in the immediate TGF-β family, designated as TGF-β1, TGF-β2, TGF-β3, TGF-β4, and TGF-β5, with the mammalian isoforms being TGF-β1, TGF-β2, and TGF-β3. Each isoform is encoded by a distinct gene and is expressed in tissue-specific and developmentally regulated manner. For example, TGF-β1 mRNA is broadly expressed in epithelial, endothelial, hematopoietic, and connective tissue cells, while TGF-β2 mRNA is primarily expressed in epithelial and neuronal cells, and TGF-β mRNA is primarily expressed in mesenchymal cells. The mammalian isoforms are highly conserved among species, indicating a critical biological function for each isoform. Despite their similarities, these isoforms differ in their binding affinities for TGF-β receptors.

Members of the TGF-β family initiate their cellular action by binding to three high affinity receptors designated as types I, II, and III (endoglin is another TGF-β receptor that is abundant on endothelial cells). The type III receptors (also called beta glycan), the most abundant type when present, function by binding all three TGF-β isoforms and then presents them to the signaling receptors, type I and II. The soluble extracellular domain of the type III receptor can function as a TGF-β antagonist. (Vilchis Landeros et al., Biochem. J. 355:215-222[2001]). The intracellular domains of the type I and II receptors contain serine/threonine protein kinases, which initiate intracellular signaling by phosphorylating several signal transduction proteins referred to as “SMADs” (this term was derived from the Sma and MAD gene homologues identified in Caenorhabditis elegans and Drosophila melanogaster). Although TGF-βs may bind the type III receptor, which then presents the TGF-β to the type I and II receptors, TGF-β1 and TGF-β3 are also capable of directly binding the type II receptors. Following binding of ligand to the type II receptors, the type II receptor recruits, binds, and transphosphorylates the type I receptors, thereby stimulating the protein kinase activity of the receptors. In this general manner, TGF-βs initiates signal transduction.

In terms of biological activity, TGF-β is involved in the regulation of the cell cycle, immunosuppression, tumor suppression, angiogenesis, development, and other cellular functions. In normal cells, TGF-β can act as a tumor suppressor by inhibiting cellular proliferation and/or by promoting cellular differentiation or apoptosis. During the course of tumorigenesis, many cells lose their TGF-β-mediated growth inhibition. After development of resistance to growth inhibition by TGF-β, tumor cells and stromal cells within tumors often increase their production of TGF-β. This increased TGF-β production is associated with increased invasiveness and metastasis of tumor cells to distant organs, at least partially due to TGF-β-mediated stimulation of angiogenesis, cell motility, immunosuppression, and an altered interaction of tumor cells with the extracellular matrix. Thus, tumor cell resistance to TGF-β and concomitant overproduction of the TGF-β ligand results in enhancement of tumor formation and greater aggressiveness of those tumor cells. Indeed, TGF-β and the associated receptors play a very important role in health and disease. Therefore, there remains a need in the art to interfere with metastasis associated with TGF-β, without interfering with the normal, physiological roles of TGF-β.

### SUMMARY OF THE INVENTION

The present invention provides methods and compositions for the suppression of metastasis by a soluble TGF-β antagonist (SR2F). This antagonist is composed of the soluble extracellular domain of the type II TGF-β receptor fused to the Fc domain of human IgG. In particular, the present invention is directed to the use of SR2F to prevent metastasis without affecting the normal physiological role of TGF-β. Thus, the SR2F of the present invention discriminates between “physiological” TGF-β and “pathological” TGF-β in such a manner that only the “pathological” TGF-β is affected by the administration of SR2F.

The present invention provides methods suppressing metastasis comprising administering a soluble TGF-β antagonist to a subject having at least one tumor, under conditions such that the normal functions of TGF-β are not detrimentally affected by the soluble transforming growth factor beta antagonist. In some preferred embodiments, the soluble TGF-β antagonist is SR2F. In other embodiments the soluble TGF-β antagonist is an antibody. In another embodiment the soluble TGF-β antagonist is an antisense TGF-β nucleic acid molecule. In still a further embodiment, the soluble TGF-β antagonist is a soluble dominant negative TGF-β receptor.

In yet another embodiment, the soluble TGF-β antagonist is decorin. In another embodiment, the soluble TGF-β antagonist is a beta glycan. In yet another embodiment, the soluble TGF-β antagonist is a latency associated peptide. In some particularly preferred embodiments, the SR2F differentiates between normal TGF-β and pathological TGF-β. In further embodiments, the TGF-β is TGF-β1. In another embodiment the TGF-β is TGF-β2. In yet another embodiment, TGF-β is TGF-β3. In some preferred embodiments, the subject is a human. In alternative embodiments, the tumor is selected from the group consisting of mammary tumors, prostate tumors, colon tumors, gastric tumors, liver tumors, pancreatic tumors, lung tumors, kidney tumors, bladder tumors, nasopharyngeal carcinomas, melanomas, chondrosarcomas and osteosarcomas. It yet other embodiments, the tumor is prevented from metastasizing.

The present invention also provides transgenic non-human animals comprising soluble transforming growth factor beta antagonist. In some embodiments, the soluble TGF-β antagonist is SR2F. In yet another embodiment, the soluble TGF-β antagonist is an antibody. In still another embodiment, the soluble TGF-β antagonist is an antisense TGF-β nucleic acid molecule. In still further embodiments, the soluble TGF-β antagonist is a soluble dominant negative TGF-β receptor. In another embodiment, the soluble TGF-β antagonist is decorin. In yet another embodiment, the soluble TGF-β antagonist is a beta glycan. In still another embodiment, the soluble TGF-β antagonist is a latency associated peptide. In some preferred embodiments, the soluble TGF-β antagonist prevents metastasis of tumors in the transgenic animal. In some embodiments, the non-human transgenic animal is a rodent.

The present invention additionally provides compositions comprising a container with a soluble TGF-β antagonist and instructions for use for treating cancer in a subject. In an embodiment, the soluble TGF-β antagonist is SR2F. In yet another embodiment, the soluble TGF-β antagonist is an antibody. In still another embodiment, the soluble TGF-β antagonist is an antisense TGF-β nucleic acid molecule. In still further embodiments, the soluble TGF-β antagonist is a soluble dominant negative TGF-β receptor. In another embodiment, the soluble TGF-β antagonist is decorin. In yet another embodiment, the soluble TGF-β antagonist is a beta glycan. In still another embodiment, the soluble TGF-β antagonist is a latency associated peptide.

The present invention also provides methods for treating a subject having cancer comprising the steps of: (a) administering a soluble TGF-β antagonist and (b) and administering a chemotherapeutic agent. In an embodiment of the present invention, the soluble TGF-β antagonist is SR2F. In yet another embodiment, the soluble TGF-β antagonist is an antibody. In still another embodiment, the soluble TGF-β antagonist is an antisense TGF-β nucleic acid molecule. In still further embodiments, the soluble TGF-β antagonist is a soluble dominant negative TGF-β receptor. In another embodiment, the soluble TGF-β antagonist is decorin. In yet another embodiment, the soluble TGF-β antagonist is a beta glycan. In still another embodiment, the soluble TGF-β antagonist is a latency associated peptide.

The present invention provides methods for treating a subject having cancer, comprising the step of administering a soluble TGF-β antagonist to a subject having at least one tumor, under conditions such that the normal functions of TGF-β are not detrimentally affected by said TGF-β antagonist. In an embodiment of the present invention, the TGF-β antagonist is SR2F. In yet another embodiment, the soluble TGF-β antagonist is an antibody. In still another embodiment, the soluble TGF-β antagonist is an antisense TGF-β nucleic acid molecule. In still further embodiments, the soluble TGF-β antagonist is a soluble dominant negative TGF-β receptor. In another embodiment, the soluble TGF-β antagonist is decorin. In yet another embodiment, the soluble TGF-β antagonist is a beta glycan. In still another embodiment, the soluble TGF-β antagonist is a latency associated peptide. In another embodiment, the subject is human. In still further embodiments, the tumor is selected from the group consisting of mammary tumors, prostate tumors, colon tumors, gastric tumors, liver tumors, pancreatic tumors, lung tumors, kidney tumors, bladder tumors, nasopharyngeal carcinomas, melanomas, chondrosarcomas, and osteosarcomas. In other embodiments, the tumor is prevented from metastasizing. In another embodiment, the TGF-β is TGF-βl. In yet another embodiment, the TGF-β is TGF-β2. In still another embodiment, the TGF-β is TGF-β3.

The present invention also provides methods for treating a subject having cancer comprising the steps of: (a) administering a soluble TGF-β antagonist and (b) and administering an immunotherapeutic agent. In an embodiment of the present invention, the soluble TGF-β antagonist is SR2F. In yet another embodiment, the soluble TGF-β antagonist is an antibody. In still another embodiment, the soluble TGF-β antagonist is an antisense TGF-β3 nucleic acid molecule. In still further embodiments, the soluble TGF-β antagonist is a soluble dominant negative TGF-β receptor. In another embodiment, the soluble TGF-β antagonist is decorin. In yet another embodiment, the soluble TGF-β antagonist is a beta glycan. In still another embodiment, the soluble TGF-β antagonist is a latency associated peptide.

### DESCRIPTION OF THE FIGURES

FIG. 1 provides a schematic of the SR2F soluble TGF-β antagonist comprising the extracellular domain of the type II TGF-β receptor fused to the Fc domain of the human IgG₁.

FIG. 2 provides the sequence of the SR2F construct (SEQ ID NO:1). The lower case letters indicate the small introns in the Fc sequence.

FIG. 3 provides a schematic of the SR2F transgene. In this construct, transgene expression is driven by the mammary-selective MMTV-LTR promoter/enhancer. Small introns are present in the Fc domain of the SR2F and the SV40 3′UTR.

FIG. 4 provides a graph showing the reversal of the growth inhibitory effects of TGF-β1 by transfected SR2F. MDA MB435 cells stably transfected with empty vector (pcDNA3), membrane-bound dominant negative type II TGF-β receptor (DNR) or the soluble TGF-β antagonist (SR2F) were assayed for their resistance to the growth inhibitor effects of added TGF-β1. In this graph, cell proliferation was normalized to the no TGF-β condition for each construct. Results are shown as the mean±S.D. (standard deviation) of three determinations.

FIG. 5 provides a graph showing the determination of the molar ratio of purified SR2F required for neutralization of TGF-β activity. The ability of increasing amounts of purified SR2F to reverse the growth inhibitory activity of 2 pM TGF-β1 on Mv1Lu cells was determined. The results are shown as the mean±S.D. (standard deviation) of three determinations. The molar ratio of SR2F:TGF-β1 giving 90% neutralization of biological activity is indicated by the dotted lines in this graph.

FIG. 6 provides results of a Northern blot analysis showing expression of SR2F mRNA in different tissues. RNA was prepared from a homozygous adult virgin female transgenic mouse, and the Northern blot was hybridized with a probe specific for the human Fc region of SR2F. A mammary gland from a wild-type (WT) mouse served as a negative control. The ethidium bromide-stained ribosomal bands are shown as a loading control in the lower panel.

FIG. 7 provides in situ hybridization results showing cell-specific expression of SR2F in the mammary gland. As shown, specific expression of SR2F mRNA was observed in the ductal epithelial cells (EC) of the mammary gland, but not in the cells of the fat pad (FP) or blood vessels (BV) of a virgin female transgenic mouse. The black dots indicate positive signal. No hybridization was observed with the sense control probe.

FIG. 8 provides a graph showing SR2F protein levels in serum and tissues from male and female transgenic mice. Sera and tissue extracts from 2.5 month old virgin male and female homozygous transgenic mice were assayed for SR2F protein using a specific ELISA assay. The results are shown as the mean±S.D. (standard deviation) of three mice of each sex. In this Figure, “MG” refers to mammary gland samples, and “SV” refers to seminal vesicle samples.

FIG. 9 provides a graph showing elevated SR2F levels in serum and mammary glands of parous and virgin female SR2F mice. SR2F levels were determined by ELISA assay from one multiparous and three virgin female homozygous SRF mice aged 12 to 14 months.

FIG. 10 provides results of a Western blot analysis of SR2F in mammary gland extracts. Extracts of wild-type (WT) and transgenic (TG) mammary glands were probed for the presence of SR2F using an anti-human Fc domain antibody. Purified SR2F was used as a positive control. Band specificity was demonstrated by pre-blocking the primary antibody with an excess of human IgG (huIgG).

FIG. 11 provides ligand affinity cross-linking results showing that SR2F from transgenic serum can bind TGF-β1. Sera from wild-type (WT) and transgenic (TG) mice were probed for TGF-β1 binding proteins by ligand affinity cross-linking with ¹²⁵I-TGF-β. Purified SR2F in buffered saline or purified SR2F spiked into wild-type serum was used as a positive control.

FIG. 12 provides a graph showing the determination of in vivo half-life of SR2F. Sequential bleeds were taken from wild-type offspring of a hemizygous transgenic mother, and serum SR2F levels were determined by ELISA. Maternally-transferred SR2F was the only source of SR2F in these mice. The results are show as the mean±S.D. (standard deviation) of five to ten mice at each time point.

FIG. 13 shows the effect of SR2F on the ability of the 37-32 melanoma cells to form metastases in internal organs following tail vein injection into either SR2F transgenic or wild-type (wt) mice (pilot study). All metastases were histologically confirmed.

FIG. 14 provides a graph showing that SR2F causes a dose-dependent decrease in the number of metastases in the liver. The number of metastases was determined on histological sections. Circulating SR2F levels were determined using an ELISA assay. The result for the wild-type cohort (0 ng/ml SR2F) is shown as the mean±S.D. (standard deviation) (n=4).

FIG. 15 provides a graph showing the time course of development of grossly evident metastases in the liver in a large scale study, showing the effect of SR2F. Mice were necropsied at 21, 28, and 35 days following injection with 37-32 melanoma cells and the numbers of grossly visible metastases in the livers were counted. No metastases were visible at 21 days post-injection. In this graph, “WT” refers to wild-type mice and “SR2F” refers to MMTV-SR2F transgenic mice. Statistical analysis was done using the t-test for independent samples.

FIG. 16 provides a graph showing the effect of SR2F on the incidence of histologically detectable metastases in multiple organs, 35 days after innoculation with 37-32 melanoma cells.

FIG. 17 provides data showing the relationship between the number of liver metastases and the levels of circulating SR2F. The data for the wild-type cohort (0 ng/ml SR2F) is expressed as the mean±S.D. (standard deviation) (n=9). The data represent microscopically-detected metastases in livers from the 35 day time point.

FIG. 18 provides a graph showing the effect of SR2F on tumor latency in the MMTV-Neu mammary tumor model. Latency was determined at the time to first appearance of a palpable mammary tumor. The mean time to appearance of the first tumor was 34 weeks for the Neu group and 32.5 weeks for the Neu/SR2F group.

FIG. 19 provides a graph showing the effect of SR2F on survival in the MMTV-mammary tumor model. Survival in this model was primarily determined by the size of the primary tumors, as mice must be euthanized when any primary tumor reaches 2 cm in diameter, regardless of health status. The survival curves are not statistically different (p 0.3, Log-rank test).

FIG. 20 provides histopathology results of primary mammary tumors and lung metastases in the MMTV-neu mammary tumor model. Panel A shows mammary tissue, while Panel B shows lung tissue. The arrows indicate the boundaries of the lesion.

FIG. 21 provides a graph showing the effect of SR2F on the incidence of lung metastases. Following necropsy, lungs were examined for histological evidence of metastases. Statistical analysis was done using the Fisher Exact probability test.

FIG. 22 provides a graph showing the effects of prolonged exposure to SR2F on memory T cell phenotype. The percentage of CD4+ T cells with a memory T cell phenotype (CD4⁺CD22^{high}CD62^{low}) was determined by FACS analysis of spleens from wild-type and MMTV-SR2F transgenic mice at different ages. TGF-β1 null (TGF-β1^{-/-}) and age- and strain-matched wild-type (TGF-β1^{+/+}) mice are shown for comparison. The TGF-β1 null mice do not survive beyond about 3 weeks of age.

### DESCRIPTION OF THE INVENTION

The present invention provides methods and compositions for the suppression of metastasis by a soluble TGF-β antagonist (SR2F). This antagonist is composed of the soluble extracellular domain of the type II TGF-β receptor fused to the Fc domain of human IgG. In particular, the present invention is directed to the use of SR2F to prevent metastasis without affecting the normal physiological role of TGF-β. Thus, the SR2F of the present invention discriminates between “physiological” TGF-β and “pathological” TGF-β in such a manner that only the “pathological” TGF-β is affected by the administration of SR2F to an animal. Thus, administration of SR2F to a subject with cancer results in the prevention of metastasis, yet does not impact the normal physiological functions of TGF-β (e.g., as a tumor suppressor).

In addition, the present invention provides transgenic mice (MMTV-SR2F mice) that express high levels of SR2F in their mammary glands, male sex organs and serum. These transgenic animals show no consistent pathologies and no perturbation in their phenotype. In addition, SR2F was determined to be correctly folded in vivo and was found to be capable of binding to TGF-β. These animals were used to demonstrate the effectiveness of SR2F in preventing metastasis following tail vein injection of isogeneic melanoma cell line. In addition, the MMTV-SR2F transgenic mice were crossed with MMTV-neu mice, to determine whether SR2F would impact primary tumor development or metastasis from a primary tumor in a more realistic model of metastatic cancers. It was found that although SR2F still suppresses metastasis, primary tumor development was not affected by SR2F in these animals. Thus, these animals provide means to further assess tumor metastasis and to prevent tumors and/or metastasis of malignant cells.

A. TGF-β are Multifunctional Growth Factors with Key Roles in Normal Tissue Homeostasis and in the Pathogenesis of Many Diseases

As indicated above, there are three mammalian isoforms of TGF-β, namely TGF-β-1, 2, and 3. TGF-β1 is quantitatively the major isoform, but essentially every tissue expresses one or more of the three isoforms, together with their cognate receptors. Expression patterns of the three isoforms differs spatially and temporally, both during development and in the adult animal, indicating that they play non-redundant roles. In support of this concept, knockout mice for the three isoforms have non-overlapping spectra of phenotypes. All three TGF-βs are clearly important in development, since knocking out any of these genes causes some embryonic or perinatal lethality. Additional roles in the adult animal can be inferred from the expression patterns of the TGF-βs (both in the unperturbed animal and in response to challenge), from the phenotypes of mice in which TGF-β function has been compromised (either through genetic manipulation or the application of TGF-β antagonists), and from in vitro studies showing effects of TGF-β on different specialized cell types. Thus, TGF-βs play key roles in regulating cell proliferation, differentiation and programmed cell death, immune system function, angiogenesis, and tissue repair. Consequently, many disease processes are associated with aberrant TGF-β function. Loss of TGF-β function has been implicated in the pathogenesis of cancer, atherosclerosis and autoimmune disease, while excessive TGF-β production has been implicated in fibroproliferative disorders, in parasite-induced immunosuppression, and in metastasis (for review, see e.g., Roberts and Spom, The Transforming Growth Factors-β, in Sporn and Roberts (eds), Handbook of Experimental Pharmacology: Peptide Growth Factors and Their Receptors, Springer Verlag, Berlin [1990], at pages 419-472; Flanders and Roberts, Transforming Growth Factor-β, in Oppenheim and Feldmann, Cytokine Reference, Academic Press, London [2000]; Dunker and Krieglstein, Eur. J. Biochem., 267:6982-6988 [2000]; Branton and Kopp, Microbes Infect., 1:1349-1365 [1999]; and Chen and Wahl, Microbes Infect., 1:1367-1380 [1999]).

B. TGF-βs Play a Complex Dual Role in Tumorigenesis by Suppressing Tumorigenesis in Early Stages of Tumor Development, But Promoting it in the Later, More Advanced Stages

TGF-βs are potent inhibitors of epithelial cell proliferation, and the TGF-β system has tumor suppressor activity in many tissues (for review, see e.g., Gold, Crit. Rev. Oncol., 10:303-360 [1999]; Massague et al., Cell, 103:295-309 [2000]; and Akhurst and Balmain, J. Pathol., 187:82-90 [1999]). Reduction or loss of TGF-β receptors or downstream signaling components is observed in many human tumor types, including tumors of the gastrointestinal tract, breast and prostate. Studies using genetically-engineered mouse models or xenografts of genetically manipulated tumor cell lines have confirmed a causal connection between diminished TGF-β function and increased tumorigenesis. However, the role of TGF-βs in tumorigenesis is complex, as many late-stage human tumors show increased expression of TGF-β, which is associated with increased metastasis and poor prognosis. It appears that TGF-βs function as tumor suppressors early in tumorigenesis, but that in the later stages, they may function as oncogenes and promote the development of aggressive metastatic disease. The mechanism for promotion of metastasis is thought to include enhanced tumor cell invasiveness, enhanced angiogenesis and suppression of the immune surveillance system. TGF-β1 is the isoform that is most commonly upregulated in late-stage human cancer, though TGF-β2 and TGF-β3 have been implicated in some instances.

C. Correlative Evidence for TGF-β's Pro-Metastatic Role in Advanced Human Cancers

TGF-β expression is increased in many advanced human cancers and is correlated with enhanced invasion and/or metastasis. TGF-β1 and TGF-β3 are the isoforms that are usually involved. Frequently, plasma levels of the TGF-βs are also increased in cancer patients with advanced disease, indicating that the tumors are secreting significant amounts of TGF-β into the circulation. Tumors showing elevated TGF-β expression include breast, colon, gastric, liver, pancreatic, prostate, lung, kidney, bladder and nasopharyngeal carcinomas, melanomas, chondrosarcomas and osteosarcomas.

1. Breast Cancer

Immunohistochemical staining for TGF-β1 associates with disease progression in human breast cancer (Gorsch et al., Canc. Res., 52:6949-6952 [1992]), and correlates with node positivity and metastasis (Walker and Dearing, Eur. J. Canc., 28:641-644 [1992]). Secreted extracellular TGF-β1 protein is increased at the advancing edge of primary human breast carcinomas and in lymph node metastases (Dalal et al., Am. J. Pathol., 143:381-389 [1993]). TGF-β1 is increased in the plasma of 81% newly-diagnosed breast cancer patients, and levels are normalized by surgical resection in node negative patients, but not in node positive patients, suggesting that primary tumors and metastases secrete significant quantities of TGF-β1 into the circulation (Kong et al., Ann. Surg., 222:155-162 [1995]). Increased plasma levels of TGF-β3 have also been found in breast cancer patients with positive lymph nodes (Li et al., Intl. J. Canc., 79:455-459 [1998]), and the combination of lymph node involvement and positive TGF-β3 expression in the invasive tumor has been associated with poor prognosis (Ghellal et al., Anticanc. Res., 20:4413-4418 [2000]).

2. Cancer of the Gastrointestinal Tract

For colon cancer patients, intense staining for TGF-β1 in the resected primary tumor has been significantly correlated with disease progression to metastasis (Friedman et al., Canc. Epidemiol. Biomarkers Prev., 4:549-554 [1995]). In addition, increased levels of TGF-β1 staining have been found in the cancer cells invading local lymph nodes when compared with the primary tumor, and elevated TGF-β1 was implicated in the metastatic process in 75% of the cases examined (Picon et al., Canc. Epidemiol. Biomarkers Prev., 7:497-504 [1998]). Plasma TGF-β1 and TGF-β2 levels are increased in patients with colorectal cancer and levels are higher in more advanced disease (Tsushima et al., Gastroenterol., 110:375-382 [1996]; and Bellone et al., Eur. J. Canc., 37:224-233 [2001]). Similarly, elevated plasma TGF-β1 levels were seen in patients with hepatocellular carcinoma, and levels were normalized following resection of the tumor, indicating that the tumor was the source of the TGF-β1 (Shirai et al., Jpn. Canc. Res., 83:676-679 [1992]). Positive staining for TGF-β1 in gastric cancer tissues is closely related to serosal invasion and lymph node metastasis (Maehara et al., J. Clin. Oncol., 17:607-614 [1999]), and elevated serum levels of TGF-β1 correlate with lymph node metastasis and poor prognosis (Saito et al., Anticanc. Res., 20:4489-4493 [2000]). In addition, mRNAs for TGF-β1, 2 and 3 are increased in 50% of pancreatic cancer cases and the increased expression correlates with decreased survival (Friess et al., Gastroenterol., 105:1846-1856 [1993]).

3. Prostate Cancer

Increased TGF-β1 staining is associated with higher tumor grade and metastasis in prostate cancer patients (Wikstrom et al., Prostate 37:19-29 [1998]). Increased TGF-β1 staining is a negative predictive factor for patient survival (Stravodimos et al., Anticanc. Res., 20:3823-3828 [2000]). Primary tumors that had metastasized have shown higher levels of staining for TGF-β1 than those that had not metastasized (Eastham et al., Lab. Invest., 73:628-635 [1995]). Furthermore, plasma TGF-β1 levels are significantly elevated in patients with clinically evident metastases (Adler et al., J. Urol., 161:182-187 [1999]), or with primary stage III/IV disease (Ivanovic et al., Nat. Med., 1:282-284 [1995]).

4. Other Tumor Types

Increased extractable TGF-β1 protein was found in the primary tumors of lung cancer patients with lymph node metastasis compared with those without metastasis (Hasegawa et al., Canc., 91:964-971 [2001]). Elevated plasma levels of TGF-β31, and to a lesser extent TGF-β2, are found in melanoma patients with disseminated but not loco-regional disease (Krasagakis et al., Br. J. Canc., 77:1492-1494 [1998]). In osteosarcomas, elevated immunohistochemical staining for TGF-β1 or TGF-β3 is associated with a higher rate of subsequent lung metastasis (Yang et al., J. Exp. Med., 184:133-142 [1998]). Plasma TGF-β1 levels are also significantly elevated in patients with chondrosarcomas (Gridley et al., Canc. Detect. Prev., 22:20-29 [1998]), and renal cell carcinomas (Wunderlich et al., Urol. Intl., 60:205-207 [1998]; and Junker et al., Cytokine 8:794-798 [1996]), suggesting that these types of tumors secrete high levels of TGF-β. Serum TGF-β1 levels are increased in patients with invasive but not superficial bladder cancer, although no further increase is found in patients with metastatic disease (Eder et al., J. Urol., 156:953-957 [1996]). Serum TGF-β1 is also increased in patients with Epstein-Barr virus-associated nasopharyngeal carcinoma, particularly in patients with relapsing tumors (Xu et al., Intl. J. Canc., 84:396-399 [1999]).

D. Experimental Evidence that TGF-β is a Pro-Metastatic Factor

In a number of tumor cell model systems, pretreatment with purified TGF-β or transfection with TGF-β1 cDNA results in an increase in metastatic potential. Conversely, blocking the tumor cell responsiveness to TGF-β or neutralizing TGF-β production decreases metastatic efficiency in vivo. This strongly suggests that TGF-β can promote metastasis. Possible mechanisms for which evidence has been obtained include: (i) suppression of immune surveillance; (ii) promotion of invasiveness and motility; and (iii) promotion of angiogenesis. However, an understanding of the mechanisms is not necessary in order to use the present invention. Indeed, it is not intended that the present invention be limited to any particular mechanism(s).

1. Experimental Addition of TGF-β Promotes Metastasis

Pretreatment in serum-free culture of a rat mammary adenocarcinoma cell line with TGF-β1 protein was found to cause a significant increase in the number of lung metastases following injection into syngeneic rats (Welch et al., Proc. Natl. Acad. Sci. USA, 87:7678-7682 [1990]). Transfection of primary human prostate tumor cells with the TGF-β1 gene was found to stimulate metastasis after orthotopic implantation in SCID mice (Stearns et al., Canc. Res., 5:711-720 [1999]). Similar results were obtained with rat prostate cancer cells (Steiner and Barrack, Mol. Endocrinol., 6:15-25 [1992]) and Chinese hamster ovary cells (Ueki et al., Jpn. J. Canc. Res., 84:589-593 [1993]).

2. Experimentally Decreasing TGF-β Production or Activity Reduces Metastasis

Treatment of athymic mice with neutralizing antibodies to TGF-β1, 2, and 3 has been found to suppress the formation of lung metastases following intraperitoneal inoculation with the human breast cancer cell line MDA-MB-231 (Arteaga et al., J. Clin. Invest., 92:2569-2576 [1993]). The same antibody caused a three-fold decrease in the number of metastases formed when B16F1 melanoma cells were injected into the tail vein of syngeneic mice (Wojtowicz-Praga et al., J. Immunother. Emphasis Tumor Immunol., 19:169-175 [1996]). In other reports, an anti-TGF-β1 monoclonal antibody was found to decrease the development of metastases following subcutaneous implantation of human carcinoma cell lines into athymic mice (Hoefer and Anderer, Canc. Immunol. Immunother., 41:302-308 [1995]). In all three of these studies, suppressive effects of TGF-β on immunosurveillance by natural killer cells, monocytes or lymphokine-activated killer cells of the host animal were implicated in the increased metastatic efficiency. In addition, treatment of malignant mouse fibrosarcoma cells with specific antisense oligonucleotides to TGF-β1 significantly decreased the metastatic properties of these cells, suggesting that TGF-β produced by the tumor cell itself is important in promoting metastasis (Spearman et al., Gene 149:25-29 [1994]).

3. Reduction of Tumor Cell Responsiveness to TGF-β can Reduce Metastasis

In three different experimental systems, interfering with the responsiveness of a mammary tumor cell line to TGF-β by transfection with a dominant negative type II TGF-β receptor has caused a significant decrease in the metastatic efficiency of these cells (McEarchem et al., Int. J. Canc., 91:76-82 [2001]; Oft et al., Curr. Biol., 8:1243-1252 [1998]; and Yin et al., J. Clin. Invest., 103:197-206 [1999]). In the case of the human breast cancer cell line MDA-MB-23 1, bony metastases were significantly reduced and survival was prolonged in a xenograft model using athymic mice (Yin et al., supra). These results suggest that, at least in breast cancer, TGF-β acting directly on the tumor cell can increase metastatic efficiency. Mechanisms include enhanced invasiveness and increased production of parathyroid hormone-related peptide.

4. In Some Cell Types, TGF-β May Suppress Metastasis

TGF-β is not uniformly pro-metastatic however, as pretreatment with TGF-β has been reported to inhibit formation of pulmonary metastases by Chinese hamster chondrosarcoma cells (Fujisawa et al., J. Exp. Med., 187:203-213 [2000]), transfection with TGF-β3 reduced metastatic dissemination of rat oral carcinoma cell lines (Davies et al., J. Oral. Pathol. Med., 29:232-240 [2000]), and overexpression of the type II TGF-β receptor reduced the metastatic potential of K-ras-transformed thyroid cells (Turco et al., Intl. J. Canc., 80:85-91 [1999]). This suggests that the ability of TGF-β to promote metastasis may vary with tumor type.

E. Phenotypes of Mice with Compromised TGF-β Function

Since TGF-βs play such important roles in maintaining normal cellular homeostasis in many organ systems, a key conceptual problem with the use of TGF-β antagonists to treat TGF-β-driven pathologies has been the likelihood of undesired side-effects on the normal tissues, including but not limited to aberrant cell proliferation and increased tumor formation due to loss of tumor suppressor function of TGF-βs in many epithelia, as well as problems due to dysregulation of the immune system (e.g., multifocal inflammation, autoimmune manifestations and myeloid hyperplasia). These pathologies are predicted based on studies of mice with experimentally compromised TGF-β function.

1. Aberrant Proliferation and Enhanced Tumorigenesis in TGF-β Compromised Mice

TGF-β1 null mice on a Rag2 null genetic background that permits extended survival develop non-metastatic colon cancer (Engle et al., Canc. Res., 59:3379-3386 [1999]), consistent with the idea that endogenous TGF-β1 functions as a tumor suppressor in the colonic epithelium. TGF-β1+/− mice with only one functional TGF-β1 allele show hyperplasia of the glandular stomach (Boivin et al., Lab. Invest., 74:513-518 [1996]), and an increased susceptibility to carcinogen-induced tumorigenesis in the liver and lung (Tang et al., Nat. Med., 4:802-807 [1998]). Similarly, interfering with TGF-β responsiveness by targeted overexpression of a dominant negative TGF-β receptor causes hyperplasia and increased susceptibility to carcinogen-induced tumorigenesis in the skin and mammary gland (Amendt et al., Oncogene 17:25-34 [1998]; and Bottinger et al., Canc. Res., 57:5564-5570 [1997]), and an increase in spontaneous mammary tumorigenesis (Gorska et al., Proc. Am. Assoc. Canc. Res., 42:422 [2001]).

2. Immune Phenotypes in TGF-β Compromised Mice

Soon after weaning, TGF-β null mice die of a rapid wasting syndrome associated with a multifocal inflammatory response leading to massive infiltration of lymphocytes and macrophages into many organs, particularly the heart and lungs (Shull et al., Nature 359:693-699 [1992]; and Kulkarni et al., Proc. Natl. Acad. Sci USA 90:770-774 [1993]). The syndrome has many of the hallmarks of autoimmune disease, including circulating antibodies to nuclear antigens, immune complex deposition and enhanced expression of major histocompatibility complex antigens (MHCI and MHCII) (Dang et al., J. Immunol., 155:3205-3212 [1995]). In MCH deficient backgrounds in which the inflammation is suppressed, there is a myeloid hyperplasia (Letterio et al., J. Clin. Invest., 98:2109-2119 [1996]). These studies suggest key roles for TGF-β1 in maintaining normal homeostasis in multiple compartments of the immune system. Consistent with this, reduction in TGF-β responsiveness by transgenic expression of a dominant negative TGF-β receptor in CD4+ and CD8+ T-cells causes T-cell differentiation into effector T-cells which also leads to an autoimmune-like syndrome (Gorelik and Flavell, Immun., 12:171-181 [2000]), while expression of the dominant negative receptor in early T-cells gave rise to a CD8+ T cell lymphoproliferative disorder resulting in the massive expansion of the lymphoid organs (Lucas et al., J. Exp. Med., 191:1187-1196 [2000]).

F. Use of TGF-β Antagonists to Treat Disease

TGF-β antagonists (antibodies, SR2F receptor body, antisense TGF-β DNA and dominant negative TGF-β receptors) have been previously used to treat TGF-β-driven pathologies, especially fibrosis, in a number of animal model systems. However, these have generally been relatively short-term experiments, frequently involving local delivery of the antagonist, and the consequences of long-term exposure to TGF-β antagonists have not been assessed, particularly regarding tumorigenesis and immune system function.

1. First Generation TGF-β Antagonists

Overexpression of TGF-βs has been implicated in the pathogenesis of a number of diseases, particularly fibrotic disorders and late-stage cancer. Initial studies using TGF-β antagonists used anti-TGF-β antibodies or naturally-occurring TGF-β binding proteins. For example, both anti-TGF-β antibodies and the proteoglycan decorin, which is a TGF-β binding protein, have been used successfully in a rat model to protect against experimental kidney fibrosis (Border et al., Nature 360:361-364 [1992]; and Border et al, Nature 346:371-374 [1990]).

Another antagonist includes latency-associated peptide (LAP). TGF-βs that are synthesized as biologically latent complexes that must be activated before they can bind to the signaling receptor complex. Latency is conferred by non-covalent association of the cleaved precursor pro-region of the TGF-β pro-peptide with the mature TGF-β. The precursor pro-region is also known as the latency-associated peptide (LAP) and purified TGF-β1 LAP can function as an antagonist for all three TGF-β isoforms (Bottinger et al., Proc. Natl. Acad. Sci. USA, 93:5877 [1996]).

2. The SR2F TGF-β “Receptor Body” Antagonist

In general, antibody and binding protein-based antagonists have been relatively low affinity. The extracellular ligand binding domain of the type II TGF-β receptor has high affinity binding sites for TGF-β1 and TGF-β3 (O'Connor-McCourt et al., Ann. N.Y. Acad. Sci., 766:300-302 [1995]). The affinity is further increased when the soluble extracellular ligand binding domain is fused to the Fc domain of human immunoglobulin, which causes dimerization of the ligand binding domain. Addition of an Fc domain to soluble cytokine receptors also increases their in vivo half-life (Capon et al., Nature 337:525-531 [1989]). A soluble TGF-β receptor-Fc fusion protein (SR2F) has been generated in a number of labs, and has been used successfully to block or reduce liver fibrogenesis induced by dimethylnitrosamine or by ligation of the common bile duct in rats, fibrosis in an experimental glomerulo-nephritis model, radiation-induced enteropathy in mice, bleomycin-induced lung fibrosis in hamsters, and adventitial fibrosis and intimal lesion formation in a rat balloon catheter denudation model (Ueno et al, Hum. Gene Ther., 11:33-42 [2000]; George et al., Proc. Natl. Acad. Sci. USA 96:12719-12724 [1999]; Isaka et al., Kidney Intl., 55:465-475 [1999]; Zheng et al., Gastroenterol., 110:1286-1296 [2000]; Wang et al., Thorax 54:805-812 [1999]; and Smith et al., Circ. Res., 84:1212-1222 [1999]). In most cases, the SR2F antagonist was given as injections of purified protein, though in two cases it was given in a gene therapy approach by introduction of the cDNA into the muscle (Ueno et al., supra; and Isaka et al., supra). None of the authors noted untoward side-effects, but all were relatively short term studies. In addition, unlike the present invention, none of these references describe the use of SR2F as an inhibitor of “pathological” TGF-β associated with metastasis.

3. Contraindications for Antagonist Use

In situations where elevated TGF-β plays a protective role against pathological challenge, the presence of a TGF-β antagonist exacerbates the pathology. In one study, intracortical injection of SR2F was found to aggravate the volume of infarction in rats subjected to a 30-minute reversible cerebral focal ischemia, showing that endogenous TGF-β plays a neuroprotective role in excitotoxic and ischemic brain injury (Ruocco et al., J. Cereb. Blood Flow Metabol., 19:1345-1353 [1999]). This would suggest that the SR2F antagonist should not be used in patients at risk for stroke. However, in the mice described herein, it is not possible to detect SR2F in the brain, indicating that it does not cross the blood-brain barrier, and would therefore not cause a problem in the event of an ischemic episode in the brain.

4. Desirable Features of a TGF-β Antagonist

An ideal TGF-β antagonist has a high affinity for TGF-βs, is stable in vivo and in vitro for long-term use, and is capable in some way of discriminating between “pathological” TGF-β that is involved in causing or exacerbating a disease process, and “physiological” TGF-β that is involved in the maintenance of normal homeostasis and cellular function in multiple organ systems.

5. Extensive Activation of Latent TGF-β in Pathological Situations: a Window of Opportunity

TGF-β is synthesized in a biologically latent form that must be activated before the TGF-β can bind to the receptor and elicit a biological response (Munger et al., Kidney Intl., 51:1376-1382 [1997]). Relatively little is known about the mechanism and circumstances of TGF-β activation in vivo, due to difficulties in discriminating between and experimentally quantitating active and latent TGF-β. Using an immunofluorescence technique that distinguishes active and latent TGF-β in frozen tissue sections, it has recently been shown for the mammary gland, that activation of latent TGF-β may occur very locally on a cell-by-cell basis in epithelium of the normal tissue (Barcellos-Hoff and Ewan, Breast Canc. Res., 2:92-99 [2000]). In contrast, in the face of pathologic challenge, there may be much more widespread activation of latent TGF-β. For example, irradiation of the mammary gland caused extensive activation of TGF-β both in the epithelium, the peri-epithelial stroma and the adipose stroma (Barcellos-Hoff et al., J. Clin. Invest., 93:892-899 [1994]). Similarly, the majority of normal cells in culture secrete predominantly latent TGF-β, though cells from more advanced tumors secrete higher amounts of active TGF-β. Significantly, in studies with oncogene-transformed fibrosarcoma cell lines, the highly metastatic fibrosarcomas were distinguished by secreting a much higher fraction of the TGF-β in the active form, although all transformed lines secreted high levels of total TGF-β (Schwarz et al., Growth Factors 3:115-127 [1990]). Although an understanding of the mechanism(s) is not necessary in order to use the present invention, it is contemplated that in one embodiment of the present invention, if TGF-β is required to maintain normal homeostasis is activated locally at the site of production and binds rapidly to nearby receptors without being released from the cell, while pathological processes are associated with more widespread activation of TGF-β, then a relatively bulky antagonist like the SR2F which has no cell surface binding domains may have poor access to the cell-associated “physiological TGF-β,” but be capable of effectively neutralizing the “pathological” TGF-β. However, it is not intended that the present invention be limited to any particular mechanism(s).

G. Pharmaceutical Compositions Containing Nucleic Acid, Polypeptides, and Analogs

The present invention further provides pharmaceutical compositions which may comprise all or portions of polynucleotide sequences, polypeptides, inhibitors or antagonists, including antibodies, alone or in combination with at least one other agent, such as a stabilizing compound, and may be administered in any sterile, biocompatible pharmaceutical carrier, including, but not limited to, saline, buffered saline, dextrose, and water.

The methods of the present invention find use in treating diseases or altering physiological states. Soluble TGF-β antagonists can be administered to the patient intravenously in a pharmaceutically acceptable carrier such as physiological saline. Standard methods for intracellular delivery of peptides can be used (e.g., delivery via liposome). Such methods are well known to those of ordinary skill in the art. The formulations of this invention are useful for parenteral administration, such as intravenous, subcutaneous, intramuscular, and intraperitoneal.

Therapeutic administration of a polypeptide intracellularly can also be accomplished using gene therapy. It is contemplated that the antagonists can be administered utilizing viral vectors, liposome-encapsulated DNA or naked DNA to deliver the cDNA encoding the antagonist to target organs such as the liver or muscle, from which the newly synthesized antagonist protein would be secreted into the circulation. (Sakamoto et al., Gene Therapy, 7:1915-1924 [2000]; Isaka et al., Kidney International, 55:465-475 [1999]).

As is well known in the medical arts, dosages for any one patient depends upon many factors, including the patient's size, body surface area, age, the particular compound to be administered, sex, time and route of administration, general health, and interaction with other drugs being concurrently administered.

Accordingly, in some embodiments of the present invention, soluble TGF-β antagonists can be administered to a patient alone, or in combination with other nucleotide sequences, drugs or hormones or in pharmaceutical compositions where it is mixed with excipient(s) or other pharmaceutically acceptable carriers. In one embodiment of the present invention, the pharmaceutically acceptable carrier is pharmaceutically inert. In another embodiment of the present invention, soluble TGF-β antagonists may be administered alone to individuals subject to or suffering from a cancer.

Depending on the condition being treated, these pharmaceutical compositions may be formulated and administered systemically or locally. Techniques for formulation and administration may be found in the latest edition of “Remington's Pharmaceutical Sciences” (Mack Publishing Co, Easton Pa.). Suitable routes may, for example, include oral or transmucosal administration; as well as parenteral delivery, including intramuscular, subcutaneous, intramedullary, intrathecal, intraventricular, intravenous, intraperitoneal, or intranasal administration.

For injection, the pharmaceutical compositions of the invention may be formulated in aqueous solutions, preferably in physiologically compatible buffers such as Hanks' solution, Ringer's solution, or physiologically buffered saline. For tissue or cellular administration, penetrants appropriate to the particular barrier to be permeated are used in the formulation. Such penetrants are generally known in the art.

In other embodiments, the pharmaceutical compositions of the present invention can be formulated using pharmaceutically acceptable carriers well known in the art in dosages suitable for oral administration. Such carriers enable the pharmaceutical compositions to be formulated as tablets, pills, capsules, liquids, gels, syrups, slurries, suspensions and the like, for oral or nasal ingestion by a patient to be treated.

Pharmaceutical compositions suitable for use in the present invention include compositions wherein the active ingredients are contained in an effective amount to achieve the intended purpose. Determination of effective amounts is well within the capability of those skilled in the art, especially in light of the disclosure provided herein.

In addition to the active ingredients these pharmaceutical compositions may contain suitable pharmaceutically acceptable carriers comprising excipients and auxiliaries which facilitate processing of the active compounds into preparations which can be used pharmaceutically. The preparations formulated for oral administration may be in the form of tablets, dragees, capsules, or solutions.

The pharmaceutical compositions of the present invention may be manufactured in a manner that is itself known (e.g., by means of conventional mixing, dissolving, granulating, dragee-making, levigating, emulsifying, encapsulating, entrapping or lyophilizing processes).

Pharmaceutical formulations for parenteral administration include aqueous solutions of the active compounds in water-soluble form. Additionally, suspensions of the active compounds may be prepared as appropriate oily injection suspensions. Suitable lipophilic solvents or vehicles include fatty oils such as sesame oil, or synthetic fatty acid esters, such as ethyl oleate or triglycerides, or liposomes. Aqueous injection suspensions may contain substances which increase the viscosity of the suspension, such as sodium carboxymethyl cellulose, sorbitol, or dextran. Optionally, the suspension may also contain suitable stabilizers or agents which increase the solubility of the compounds to allow for the preparation of highly concentrated solutions.

Pharmaceutical preparations for oral use can be obtained by combining the active compounds with solid excipient, optionally grinding a resulting mixture, and processing the mixture of granules, after adding suitable auxiliaries, if desired, to obtain tablets or dragee cores. Suitable excipients are carbohydrate or protein fillers such as sugars, including lactose, sucrose, mannitol, or sorbitol; starch from corn, wheat, rice, potato, etc; cellulose such as methyl cellulose, hydroxypropylmethyl-cellulose, or sodium carboxymethylcellulose; and gums including arabic and tragacanth; and proteins such as gelatin and collagen. If desired, disintegrating or solubilizing agents may be added, such as the cross-linked polyvinyl pyrrolidone, agar, alginic acid or a salt thereof such as sodium alginate.

Dragee cores are provided with suitable coatings such as concentrated sugar solutions, which may also contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for product identification or to characterize the quantity of active compound, (i.e., dosage).

Pharmaceutical preparations which can be used orally include push-fit capsules made of gelatin, as well as soft, sealed capsules made of gelatin and a coating such as glycerol or sorbitol. The push-fit capsules can contain the active ingredients mixed with a filler or binders such as lactose or starches, lubricants such as talc or magnesium stearate, and, optionally, stabilizers. In soft capsules, the active compounds may be dissolved or suspended in suitable liquids, such as fatty oils, liquid paraffin, or liquid polyethylene glycol with or without stabilizers.

Compositions comprising a compound of the invention formulated in a pharmaceutical acceptable carrier may be prepared, placed in an appropriate container, and labeled for treatment of an indicated condition.

The pharmaceutical composition may be provided as a salt and can be formed with many acids, including but not limited to hydrochloric, sulfuric, acetic, lactic, tartaric, malic, succinic, etc. Salts tend to be more soluble in aqueous or other protonic solvents that are the corresponding free base forms. In other cases, the preferred preparation may be a lyophilized powder in 1 mM-50 mM histidine, 0.1%-2% sucrose, 2%-7% mannitol at a pH range of 4.5 to 5.5 that is combined with buffer prior to use.

For any compound used in the method of the invention, the therapeutically effective dose can be estimated initially from cell culture assays. Then, preferably, dosage can be formulated in animal models (particularly murine models) to achieve a desirable circulating concentration range that reduces pathological TGF-β levels.

A therapeutically effective dose refers to that amount of soluble TGF-β antagonists which ameliorates symptoms of the disease state. Toxicity and therapeutic efficacy of such compounds can be determined by standard pharmaceutical procedures in cell cultures or experimental animals, e.g., for determining the LD₅₀ (the dose lethal to 50% of the population) and the ED₅₀ (the dose therapeutically effective in 50% of the population). The dose ratio between toxic and therapeutic effects is the therapeutic index, and it can be expressed as the ratio LD₅₀/ED₅₀. Compounds which exhibit large therapeutic indices are preferred. The data obtained from these cell culture assays and additional animal studies can be used in formulating a range of dosage for human use. The dosage of such compounds lies preferably within a range of circulating concentrations that include the ED₅₀ with little or no toxicity. The dosage varies within this range depending upon the dosage form employed, sensitivity of the patient, and the route of administration.

The exact dosage is chosen by the individual physician in view of the patient to be treated. Dosage and administration are adjusted to provide sufficient levels of the active moiety or to maintain the desired effect. Additional factors which may be taken into account include the severity of the disease state; age, weight, and gender of the patient; diet, time and frequency of administration, drug combination(s), reaction sensitivities, and tolerance/response to therapy. Long acting pharmaceutical compositions might be administered every 3 to 4 days, every week, or once every two weeks depending on half-life and clearance rate of the particular formulation.

Normal dosage amounts may vary from 0.1 to 100,000 micrograms, up to a total dose of about 1 g, depending upon the route of administration. Guidance as to particular dosages and methods of delivery is provided in the literature (See, U.S. Pat. Nos. 4,657,760; 5,206,344; or 5,225,212, all of which are herein incorporated by reference). Those skilled in the art will employ different formulations. Administration to the bone marrow may necessitate delivery in a manner different from intravenous injections.

H. Soluble TGF-β Antagonist Therapy in Combination with Other Therapies

1. In Combination with Chemotherapy

The present invention includes use of cytototoxic chemotherapy in conjunction with treatment with soluble TGF-β antagonists. Embodiments include using cell-cycle active agents, (e.g., 5-fluorouracil) which show dose-limiting toxicity in tissue compartments with actively cycling cells, such as the bone marrow and gut. While an understanding of the mechanisms is not necessary in order to use the present invention, TGF-β keeps stem cells in a state of quiescence. The administration of a soluble TGF-β antagonist after a round of chemotherapy is contemplated to enhance stem cell proliferation and, thus, hematopoietic recovery (Sitnicka et al., Blood, 88:82-88 [1996]). Combination therapy with a soluble TGF-β antagonist and a chemotherapeutic agent leads to diminished toxicity of the chemotherapeutic agent in addition to the independently therapeutic effect of the TGF-β antagonist.

2. In Combination with Immunotherapy.

The present invention also includes treatment with soluble TGF-β antagonists in conjunction with immunotherapies. While an understanding of the mechanisms is not necessary in order to use the present invention, it is contemplated that secretion by tumors of inhibitors of the immune system limit the efficacy of immunotherapy approaches aimed at enhancing the immune recognition and destruction of the tumor (de Visser and Kast, Leukemia, 13:1188-1199 [1999]). TGF-β is an immunosuppressive agent that is highly secreted by tumors. Embodiments of the present invention include use of a TGF-β antagonist in combination with immunotherapy approaches (e.g., anti-tumor vaccination, adoptive immunotherapy) which result in a synergism between the anti-metastatic effects of the TGF-β antagonists and an enhanced efficacy of the immunotherapy.

I. Gene Therapy Using Soluble TGF-β Antagonists

The present invention also provides methods and compositions suitable for gene therapy to express or produce soluble TGF-β antagonists intracellularly. Thus, the methods described below are generally applicable across many species. In some embodiments, it is contemplated that the gene therapy is performed by providing a subject with a gene for a TGF-β antagonist (e.g., SR2F). Subjects in need of such therapy are identified by the methods described above. Accordingly, a method of gene therapy is to ablate the subjects monocytes (e.g., via radiation) and replace the monocytes with monocytes expressing the TGF-β antagonist via a bone marrow transplant. In some embodiments, the subjects monocytes may be harvested prior to radiation treatment, transfected with a vector (described below) encoding TGF-β antagonist monocytes, amplified through in vitro cultured, and reintroduced into the subject.

Viral vectors commonly used for in vivo or ex vivo targeting and therapy procedures are DNA-based vectors and retroviral vectors. Methods for constructing and using viral vectors are known in the art (See e.g., Miller and Rosman, BioTech., 7:980-990 [1992]). Preferably, the viral vectors are replication defective, that is, they are unable to replicate autonomously in the target cell. In general, the genome of the replication defective viral vectors that are used within the scope of the present invention lack at least one region that is necessary for the replication of the virus in the infected cell. These regions can either be eliminated (in whole or in part), or be rendered non-functional by any technique known to a person skilled in the art. These techniques include the total removal, substitution (by other sequences, in particular by the inserted nucleic acid), partial deletion or addition of one or more bases to an essential (for replication) region. Such techniques may be performed in vitro (i.e., on the isolated DNA) or in situ, using the techniques of genetic manipulation or by treatment with mutagenic agents.

Preferably, the replication defective virus retains the sequences of its genome that are necessary for encapsidating the viral particles. DNA viral vectors include an attenuated or defective DNA viruses, including, but not limited to, herpes simplex virus (HSV), papillomavirus, Epstein Barr virus (EBV), adenovirus, adeno-associated virus (AAV), and the like. Defective viruses, that entirely or almost entirely lack viral genes, are preferred, as defective virus is not infective after introduction into a cell. Use of defective viral vectors allows for administration to cells in a specific, localized area, without concern that the vector can infect other cells. Thus, a specific tissue can be specifically targeted. Examples of particular vectors include, but are not limited to, a defective herpes virus 1 (HSV1) vector (Kaplitt et al., Mol. Cell. Neurosci., 2:320-330 [1991]), defective herpes virus vector lacking a glycoprotein L gene (See e.g., Patent Publication RD 371005 A), or other defective herpes virus vectors (See e.g., WO 94/21807; and WO 92/05263); an attenuated adenovirus vector, such as the vector described by Stratford-Perricaudet et al. (J. Clin. Invest., 90:626-630 [1992]; See also, La Salle et al., Science 259:988-990 [1993]); and a defective adeno-associated virus vector (Samulski et al., J. Virol., 61:3096-3101 [1987]; Samulski et al., J. Virol., 63:3822-3828 [1989]; and Lebkowski et al., Mol. Cell. Biol., 8:3988-3996 [1988]).

Preferably, for in vivo administration, an appropriate immunosuppressive treatment is employed in conjunction with the viral vector (e.g., adenovirus vector), to avoid immuno-deactivation of the viral vector and transfected cells. For example, immunosuppressive cytokines, such as interleukin-12 (IL-12), interferon-gamma (IFN-γ), or anti-CD4 antibody, can be administered to block humoral or cellular immune responses to the viral vectors. In addition, it is advantageous to employ a viral vector that is engineered to express a minimal number of antigens.

In a preferred embodiment, the vector is an adenovirus vector. Adenoviruses are eukaryotic DNA viruses that can be modified to efficiently deliver a nucleic acid of the invention to a variety of cell types. Various serotypes of adenovirus exist. Of these serotypes, preference is given, within the scope of the present invention, to type 2 or type 5 human adenoviruses (Ad 2 or Ad 5), or adenoviruses of animal origin (See e.g., WO94/26914). Those adenoviruses of animal origin that can be used within the scope of the present invention include adenoviruses of canine, bovine, murine (e.g., Mavl, Beard et al., Virol., 75-81 [1990]), ovine, porcine, avian, and simian (e.g., SAV) origin. Preferably, the adenovirus of animal origin is a canine adenovirus, more preferably a CAV2 adenovirus (e.g. Manhattan or A26/61 strain (ATCC VR-800)).

Preferably, the replication defective adenoviral vectors of the invention comprise the ITRs, an encapsidation sequence and the nucleic acid of interest. Still more preferably, at least the E1 region of the adenoviral vector is non-functional. The deletion in the E1 region preferably extends from nucleotides 455 to 3329 in the sequence of the Ad5 adenovirus (PvuII-BglII fragment) or 382 to 3446 (HinfII-Sau3A fragment). Other regions may also be modified, in particular the E3 region (e.g., WO95/02697), the E2 region (e.g., WO94/28938), the E4 region (e.g., WO94/28152, WO94/12649 and WO95/02697), or in any of the late genes L1-L5.

In a preferred embodiment, the adenoviral vector has a deletion in the E1 region (Ad 1.0). Examples of E1-deleted adenoviruses are disclosed in EP 185,573, the contents of which are incorporated herein by reference. In another preferred embodiment, the adenoviral vector has a deletion in the E1 and E4 regions (Ad 3.0). Examples of E1/E4-deleted adenoviruses are disclosed in WO95/02697 and WO96/22378. In still another preferred embodiment, the adenoviral vector has a deletion in the E1 region into which the E4 region and the nucleic acid sequence are inserted.

The replication defective recombinant adenoviruses according to the invention can be prepared by any technique known to the person skilled in the art (see e.g., Levrero et al., Gene 101:195 [1991];EP 185 573; and Graham, EMBO J., 3:2917 [1984]). In particular, they can be prepared by homologous recombination between an adenovirus and a plasmid which carries, inter alia, the DNA sequence of interest. The homologous recombination is accomplished following co-transfection of the adenovirus and plasmid into an appropriate cell line. The cell line that is employed should preferably (i) be transformable by the elements to be used, and (ii) contain the sequences that are able to complement the part of the genome of the replication defective adenovirus, preferably in integrated form in order to avoid the risks of recombination. Examples of cell lines that may be used are the human embryonic kidney cell line 293 (Graham et al, J. Gen. Virol., 36:59 [1977]), which contains the left-hand portion of the genome of an Ad5 adenovirus (12%) integrated into its genome, and cell lines that are able to complement the E1 and E4 functions, as described in applications WO94/26914 and WO95/02697. Recombinant adenoviruses are recovered and purified using standard molecular biological techniques, that are well known to one of ordinary skill in the art.

The adeno-associated viruses (AAV) are DNA viruses of relatively small size that can integrate, in a stable and site-specific manner, into the genome of the cells that they infect. They are able to infect a wide spectrum of cells without inducing any effects on cellular growth, morphology or differentiation, and they do not appear to be involved in human pathologies. The AAV genome has been cloned, sequenced and characterized. It encompasses approximately 4700 bases and contains an inverted terminal repeat (ITR) region of approximately 145 bases at each end, which serves as an origin of replication for the virus. The remainder of the genome is divided into two essential regions that carry the encapsidation functions: the left-hand part of the genome, that contains the rep gene involved in viral replication and expression of the viral genes; and the right-hand part of the genome, that contains the cap gene encoding the capsid proteins of the virus.

The use of vectors derived from the AAVs for transferring genes in vitro and in vivo has been described (See e.g., WO 91/18088; WO 93/09239; US Pat. No. 4,797,368; US Pat. No., 5,139,941; and EP 488 528, all of which are herein incorporated by reference). These publications describe various AAV-derived constructs in which the rep and/or cap genes are deleted and replaced by a gene of interest, and the use of these constructs for transferring the gene of interest in vitro (into cultured cells) or in vivo (directly into an organism). The replication defective recombinant AAVs according to the invention can be prepared by co-transfecting a plasmid containing the nucleic acid sequence of interest flanked by two AAV inverted terminal repeat (ITR) regions, and a plasmid carrying the AAV encapsidation genes (rep and cap genes), into a cell line that is infected with a human helper virus (for example an adenovirus). The AAV recombinants that are produced are then purified by standard techniques.

In another embodiment, the soluble TGF-β antagonist encoding nucleic acid sequence can be introduced in a retroviral vector (e.g., as described in U.S. Pat. Nos. 5,399,346, 4,650,764, 4,980,289 and 5,124,263; all of which are herein incorporated by reference; Mann et al., Cell 33:153 [1983]; Markowitz et al., J. Virol., 62:1120 [1988]; PCT/US95/14575; EP 453242; EP178220; Bernstein et al. Genet. Eng., 7:235 [1985]; McCormick, BioTechnol., 3:689 [1985]; WO 95/07358; and Kuo et al., Blood 82:845 [1993]). The retroviruses are integrating viruses that infect dividing cells. The retrovirus genome includes two LTRs, an encapsidation sequence and three coding regions (gag, pol and env). In recombinant retroviral vectors, the gag, pol and env genes are generally deleted, in whole or in part, and replaced with a heterologous nucleic acid sequence of interest. These vectors can be constructed from different types of retrovirus, such as, HIV, MoMuLV (“murine Moloney leukaemia virus” MSV (“murine Moloney sarcoma virus”), HaSV (“Harvey sarcoma virus”); SNV (“spleen necrosis virus”); RSV (“Rous sarcoma virus”) and Friend virus. Defective retroviral vectors are also disclosed in WO95/02697.

In general, in order to construct recombinant retroviruses containing a nucleic acid sequence, a plasmid is constructed that contains the LTRs, the encapsidation sequence and the coding sequence. This construct is used to transfect a packaging cell line, which cell line is able to supply in trans the retroviral functions that are deficient in the plasmid. In general, the packaging cell lines are thus able to express the gag, pol and env genes. Such packaging cell lines have been described in the prior art, in particular the cell line PA317 (U.S. Pat. No. 4,861,719, herein incorporated by reference), the PsiCRIP cell line (See, WO90/02806), and the GP+envAm-12 cell line (See, WO89/07150). In addition, the recombinant retroviral vectors can contain modifications within the LTRs for suppressing transcriptional activity as well as extensive encapsidation sequences that may include a part of the gag gene (Bender et al., J. Virol., 61:1639 [1987]). Recombinant retroviral vectors are purified by standard techniques known to those having ordinary skill in the art.

Alternatively, the vector can be introduced in vivo by lipofection. For the past decade, there has been increasing use of liposomes for encapsulation and transfection of nucleic acids in vitro. Synthetic cationic lipids designed to limit the difficulties and dangers encountered with liposome mediated transfection can be used to prepare liposomes for in vivo transfection of a gene encoding a marker (Felgner et. al., Proc. Natl. Acad. Sci. USA 84:7413-7417 [1987]; See also, Mackey, et al., Proc. Natl. Acad. Sci. USA 85:8027-8031 [1988]; Ulmer et al., Science 259:1745-1748 [1993]). The use of cationic lipids may promote encapsulation of negatively charged nucleic acids, and also promote fusion with negatively charged cell membranes (Felgner and Ringold, Science 337:387-388 [1989]). Particularly useful lipid compounds and compositions for transfer of nucleic acids are described in WO95/18863 and WO96/17823, and in U.S. Pat. No. 5,459,127, herein incorporated by reference.

Other molecules are also useful for facilitating transfection of a nucleic acid in vivo, such as a cationic oligopeptide (e.g., WO95/21931), peptides derived from DNA binding proteins (e.g., WO96/25508), or a cationic polymer (e.g., WO95/21931).

It is also possible to introduce the vector in vivo as a naked DNA plasmid. Methods for formulating and administering naked DNA to mammalian muscle tissue are disclosed in U.S. Pat. Nos. 5,580,859 and 5,589,466, both of which are herein incorporated by reference.

DNA vectors for gene therapy can be introduced into the desired host cells by methods known in the art, including but not limited to transfection, electroporation, microinjection, transduction, cell fusion, DEAE dextran, calcium phosphate precipitation, use of a gene gun, or use of a DNA vector transporter (See e.g., Wu et al., J. Biol. Chem., 267:963 [1992]; Wu and Wu, J. Biol. Chem., 263:14621 [1988]; and Williams et al., Proc. Natl. Acad. Sci. USA 88:2726 [1991]). Receptor-mediated DNA delivery approaches can also be used (Curiel et al., Hum. Gene Ther., 3:147 [1992]; and Wu and Wu, J. Biol. Chem., 262:4429 [1987]).

Definitions

The term “antagonist” refers to molecules or compounds which inhibit the action of a “native” or “natural” compound. Antagonists may or may not be homologous to these natural compounds in respect to conformation, charge or other characteristics. Thus, antagonists may be recognized by the same or different receptors that are recognized by an agonist. Antagonists may have allosteric effects which prevent the action of an agonist. Or, antagonists may prevent the function of the agonist. In contrast to the agonists, antagonistic compounds do not result in physiologic and/or biochemical changes within the cell such that the cell reacts to the presence of the antagonist in the same manner as if the natural compound was present.

The term “agonist” refers to molecules or compounds which mimic the action of a “native” or “natural” compound. Agonists may be homologous to these natural compounds in respect to conformation, charge or other characteristics. Thus, agonists may be recognized by receptors expressed on cell surfaces. This recognition may result in physiologic and/or biochemical changes within the cell, such that the cell reacts to the presence of the agonist in the same manner as if the natural compound was present.

The “non-human animals” of the invention comprise any non-human animal, including vertebrates such as rodents, non-human primates, ovines, bovines, ruminants, lagomorphs, porcines, caprines, equines, canines, felines, aves, etc. Preferred non-human animals are selected from porcines (e.g., pigs), murines (e.g., rats and mice), most preferably mice and lagomorphs (e.g., rabbits). However, it is not intended that the present invention be limited to any particular non-human animal.

The “non-human animals having a genetically engineered genotype” of the invention are preferably produced by experimental manipulation of the genome of the germline of the non-human animal. These genetically engineered non-human animals may be produced by several methods including the introduction of a “transgene” comprising nucleic acid (usually DNA) into an embryonal target cell or integration into a chromosome of the somatic and/or germ line cells of a non-human animal by way of human intervention, such as by the methods described herein. Non-human animals which contain a transgene are referred to as “transgenic non-human animals.”

A transgenic animal is an animal whose genome has been altered by the introduction of a transgene.

The term “transgene” as used herein refers to a foreign gene that is placed into an organism by introducing the foreign gene into newly fertilized eggs or early embryos. The term “foreign gene” refers to any nucleic acid (e.g., gene sequence) which is introduced into the genome of an animal by experimental manipulations and may include gene sequences found in that animal so long as the introduced gene does not reside in the same location as does the naturally-occurring gene.

As used herein, the term “vector” is used in reference to nucleic acid molecules that transfer DNA segment(s) from one cell to another. Retroviral vectors transfer RNA, which is then reverse transcribed into DNA. However, it is not intended that the present invention be limited to retroviral or any other specific vector. The term “vehicle” is sometimes used interchangeably with “vector.”

The term “expression vector” as used herein refers to a recombinant molecule containing a desired coding sequence and appropriate nucleic acid sequences necessary for the expression of the operably linked coding sequence in a particular host organism. Nucleic acid sequences necessary for expression in prokaryotes usually include a promoter, an operator (optional), and a ribosome binding site, often along with other sequences. Eukaryotic cells are known to utilize promoters, enhancers, and termination and polyadenylation signals.

The terms “in operable combination,” “in operable order,” and “operably linked” as used herein refer to the linkage of nucleic acid sequences in such a manner that a nucleic acid molecule capable of directing the transcription of a given gene and/or the synthesis of a desired protein molecule is produced. The term also refers to the linkage of amino acid sequences in such a manner so that a functional protein is produced.

The terms “promoter element” and “promoter” as used herein, refer to a DNA sequence that precedes a gene in a DNA polymer and provides a site for initiation of the transcription of the gene into mRNA.

As used herein, the term “remedial gene” refers to a gene whose expression is desired in a cell to correct an error in cellular metabolism or to kill a cancerous cell.

As used herein, the term “selectable marker” refers to the use of a gene which encodes an enzymatic activity that confers resistance to an antibiotic or drug upon the cell in which the selectable marker is expressed. Selectable markers may be “dominant”; a dominant selectable marker encodes an enzymatic activity which can be detected in any eukaryotic cell line. Examples of dominant selectable markers include the bacterial aminoglycoside 3′ phosphotransferase gene (also referred to as the neo gene) which confers resistance to the drug G418 in mammalian cells, the bacterial hygromycin G phosphotransferase (hyg) gene which confers resistance to the antibiotic hygromycin and the bacterial xanthine-guanine phosphoribosyl transferase gene (also referred to as the gpt gene) which confers the ability to grow in the presence of mycophenolic acid. Other selectable markers are not dominant in that there use must be in conjunction with a cell line that lacks the relevant enzyme activity. Examples of non-dominant selectable markers include the thymidine kinase (tk) gene that is used in conjunction with tk7 cell lines, the CAD gene that is used in conjunction with CAD-deficient cells and the mammalian hypoxanthine-guanine phosphoribosyl transferase (hprt) gene that is used in conjunction with hprf cell lines. A review of the use of selectable markers in mammalian cell lines is provided in Sambrook, J. et al., Molecular Cloning: A Laboratory Manual, 2nd ed., Cold Spring Harbor Laboratory Press, New York (1989) pp.16.9-16.15.

As used herein, the terms “complementary” or “complementarity” are used in reference to polynucleotides (i.e., a sequence of nucleotides) related by the base-pairing rules. For example, for the sequence “A-G-T,” is complementary to the sequence “T-C-A.” Complementarity may be “partial,” in which only some of the nucleic acids' bases are matched according to the base pairing rules. Or, there may be “complete” or “total” complementarity between the nucleic acids. The degree of complementarity between nucleic acid strands has significant effects on the efficiency and strength of hybridization between nucleic acid strands. This is of particular importance in amplification reactions, as well as detection in methods which depend upon binding between nucleic acids.

As used herein, the term “hybridization” is used in reference to the pairing of complementary nucleic acids. Hybridization and the strength of hybridization (i.e., the strength of the association between the nucleic acids) is impacted by such factors as the degree of complementary between the nucleic acids, stringency of the conditions involved, the Tₘ of the formed hybrid, and the G:C ratio within the nucleic acids.

As used herein, the term “Tₘ” is used in reference to the “melting temperature.” The melting temperature is the temperature at which a population of double-stranded nucleic acid molecules becomes half dissociated into single strands. The equation for calculating the Tm of nucleic acids is well known in the art. As indicated by standard references, a simple estimate of the Tₘ value may be calculated by the equation: Tₘ=81.5+0.41(% G+C), when a nucleic acid is in aqueous solution at 1 M NaCl (See e.g., Anderson and Young, Quantitative Filter Hybridization, in Nucleic Acid Hybridization (1985). Other references include more sophisticated computations which take structural as well as sequence characteristics into account for the calculation of Tₘ.

As used herein the term “stringency” is used in reference to the conditions of temperature, ionic strength, and the presence of other compounds such as organic solvents, under which nucleic acid hybridizations are conducted. With “high stringency” conditions, nucleic acid base pairing will occur only between nucleic acid fragments that have a high frequency of complementary base sequences. Thus, conditions of “weak” or “low” stringency are often required with nucleic acids that are derived from organisms that are genetically diverse, as the frequency of complementary sequences is usually less.

As used herein, the term “amplifiable nucleic acid” is used in reference to nucleic acids which may be amplified by any amplification method. It is contemplated that “amplifiable nucleic acid” will usually comprise “sample template.”

As used herein, the term “sample template” refers to nucleic acid originating from a sample which is analyzed for the presence of “target.” In contrast, “background template” is used in reference to nucleic acid other than sample template which may or may not be present in a sample. Background template is most often inadvertent. It may be the result of carryover, or it may be due to the presence of nucleic acid contaminants sought to be purified away from the sample.

As used herein, the term “primer” refers to an oligonucleotide, whether occurring naturally as in a purified restriction digest or produced synthetically, which is capable of acting as a point of initiation of synthesis when placed under conditions in which synthesis of a primer extension product which is complementary to a nucleic acid strand is induced, (i.e., in the presence of nucleotides and an inducing agent such as DNA polymerase and at a suitable temperature and pH). The primer is preferably single stranded for maximum efficiency in amplification, but may alternatively be double stranded. If double stranded, the primer is first treated to separate its strands before being used to prepare extension products. Preferably, the primer is an oligodeoxyribonucleotide. The primer must be sufficiently long to prime the synthesis of extension products in the presence of the inducing agent. The exact lengths of the primers will depend on many factors, including temperature, source of primer and the use of the method.

As used herein, the term “probe” refers to an oligonucleotide (i.e., a sequence of nucleotides), whether occurring naturally as in a purified restriction digest or produced synthetically, which is capable of hybridizing to another oligonucleotide of interest. Probes are useful in the detection, identification and isolation of particular gene sequences. It is contemplated that any probe used in the present invention will be labeled with any “reporter molecule,” so that is detectable in any detection system, including, but not limited to enzyme (e.g., ELISA, as well as enzyme-based histochemical assays), fluorescent, radioactive, and luminescent systems. It is further contemplated that the oligonucleotide of interest (i.e., to be detected) will be labeled with a reporter molecule. It is also contemplated that both the probe and oligonucleotide of interest will be labeled. It is not intended that the present invention be limited to any particular detection system or label.

As used herein in reference to the polymerase chain reaction, the term “target” refers to the region of nucleic acid bounded by the primers used for polymerase chain reaction. Thus, the “target” is sought to be sorted out from other nucleic acid sequences. In other embodiments, the term refers to any nucleic acid (or region of nucleic acid) of interest. A “segment” is defined as a region of nucleic acid within the target sequence.

As used herein, the term “polymerase chain reaction” (“PCR”) refers to the methods of U.S. Pat. Nos. 4,683,195, 4,683,202, and 4,965,188, all of which are hereby incorporated by reference, directed to methods for increasing the concentration of a segment of a target sequence in a mixture of genomic DNA without cloning or purification. This process for amplifying the target sequence consists of introducing a large excess of two oligonucleotide primers to the DNA mixture containing the desired target sequence, followed by a precise sequence of thermal cycling in the presence of a DNA polymerase. The two primers are complementary to their respective strands of the double stranded target sequence. To effect amplification, the mixture is denatured and the primers then annealed to their complementary sequences within the target molecule. Following annealing, the primers are extended with a polymerase so as to form a new pair of complementary strands. The steps of denaturation, primer annealing and polymerase extension can be repeated many times (i.e., denaturation, annealing and extension constitute one “cycle”; there can be numerous “cycles”) to obtain a high concentration of an amplified segment of the desired target sequence. The length of the amplified segment of the desired target sequence is determined by the relative positions of the primers with respect to each other, and therefore, this length is a controllable parameter. By virtue of the repeating aspect of the process, the method is referred to as the “polymerase chain reaction” (hereinafter “PCR”). Because the desired amplified segments of the target sequence become the predominant sequences (in terms of concentration) in the mixture, they are the to be “PCR amplified.”

With PCR, it is possible to amplify a single copy of a specific target sequence in genomic DNA to a level detectable by several different methodologies (e.g., hybridization with a labeled probe; incorporation of biotinylated primers followed by avidin-enzyme conjugate detection; incorporation of ³²P-labeled deoxynucleotide triphosphates, such as dCTP or dATP, into the amplified segment). In addition to genomic DNA, any oligonucleotide sequence can be amplified with the appropriate set of primer molecules. In particular, the amplified segments created by the PCR process itself are, themselves, efficient templates for subsequent PCR amplifications.

“Amplification” is a special case of nucleic acid replication involving template specificity. It is to be contrasted with non-specific template replication (i.e., replication that is template-dependent but not dependent on a specific template). Template specificity is here distinguished from fidelity of replication (i.e., synthesis of the proper polynucleotide sequence) and nucleotide (ribo- or deoxyribo-) specificity. Template specificity is frequently described in terms of “target” specificity. Target sequences are “targets” in the sense that they are sought to be sorted out from other nucleic acid. Amplification techniques have been designed primarily for this sorting out.

Template specificity is achieved in most amplification techniques by the choice of enzyme. Amplification enzymes are enzymes that, under conditions they are used, will process only specific sequences of nucleic acid in a heterogeneous mixture of nucleic acid. For example, in the case of Qβ replicase, MDV-1 RNA is the specific template for the replicase (Kacian et al., Proc. Nat. Acad. Sci USA 69:3038 [1972]). Other nucleic acid will not be replicated by this amplification enzyme. Similarly, in the case of T7 RNA polymerase, this amplification enzyme has a stringent specificity for its own promoters (Chamberlin et al., Nature 228:227 [1970]). In the case of T4 DNA ligase, the enzyme will not ligate the two oligonucleotides where there is a mismatch between the oligonucleotide substrate and the template at the ligation junction (Wu and Wallace, Genomics 4:560 [1989]). Finally, thermostable polymerases, such as Taq and Pfu, by virtue of their ability to function at high temperature, are found to display high specificity for the sequences bounded and thus defined by the primers; the high temperature results in thermodynamic conditions that favor primer hybridization with the target sequences and not hybridization with non-target sequences.

Some amplification techniques take the approach of amplifying and then detecting target; others detect target and then amplify probe. Regardless of the approach, nucleic acid must be free of inhibitors for amplification to occur at high efficiency.

As used herein, the terms “PCR product” and “amplification product” refer to the resultant mixture of compounds after two or more cycles of the PCR steps of denaturation, annealing and extension are complete. These terms encompass the case where there has been amplification of one or more segments of one or more target sequences.

As used herein, the term “nested primers” refers to primers that anneal to the target sequence in an area that is inside the annealing boundaries used to start PCR (Mullis, et al., Cold Spring Harbor Symposia, Vol. 11, pp.263-273 [1986]). Because the nested primers anneal to the target inside the annealing boundaries of the starting primers, the predominant PCR-amplified product of the starting primers is necessarily a longer sequence, than that defined by the annealing boundaries of the nested primers. The PCR-amplified product of the nested primers is an amplified segment of the target sequence that cannot, therefore, anneal with the starting primers. Advantages to the use of nested primers include the large degree of specificity, as well as the fact that a smaller sample portion may be used and yet obtain specific and efficient amplification.

As used herein, the term “amplification reagents” refers to those reagents (deoxyribonucleoside triphosphates, buffer, etc.), needed for amplification except for pnmers, nucleic acid template and the amplification enzyme. Typically, amplification reagents along with other reaction components are placed and contained in a reaction vessel (test tube, microwell, etc.).

As used herein, the terms “restriction endonucleases” and “restriction enzymes” refer to bacterial enzymes, each of which cut double-stranded DNA at or near a specific nucleotide sequence.

As used herein, the term “recombinant DNA molecule” as used herein refers to a DNA molecule which is comprised of segments of DNA joined together by means of molecular biological techniques.

DNA molecules are said to have “5′ ends” and “3′ ends” because mononucleotides are reacted to make oligonucleotides in a manner such that the 5′ phosphate of one mononucleotide pentose ring is attached to the 3′ oxygen of its neighbor in one direction via a phosphodiester linkage. Therefore, an end of an oligonucleotides referred to as the “5′ end” if its 5′ phosphate is not linked to the 3′ oxygen of a mononucleotide pentose ring and as the “3′ end” if its 3′ oxygen is not linked to a 5′ phosphate of a subsequent mononucleotide pentose ring. As used herein, a nucleic acid sequence, even if internal to a larger oligonucleotide, also may be said to have 5′ and 3′ ends. In either a linear or circular DNA molecule, discrete elements are referred to as being “upstream” or 5′ of the “downstream” or 3′ elements. This terminology reflects the fact that transcription proceeds in a 5′ to 3′ fashion along the DNA strand. The promoter and enhancer elements which direct transcription of a linked gene are generally located 5′ or upstream of the coding region However, enhancer elements can exert their effect even when located 3′ of the promoter element and the coding region. Transcription termination and polyadenylation signals are located 3′ or downstream of the coding region.

As used herein, the term “an oligonucleotide having a nucleotide sequence encoding a gene” means a DNA sequence comprising the coding region of a gene or in other words the DNA sequence which encodes a gene product. The coding region may be present in either a cDNA or genomic DNA form. Suitable control elements such as enhancers/promoters, splice junctions, polyadenylation signals, etc. may be placed in close proximity to the coding region of the gene if needed to permit proper initiation of transcription and/or correct processing of the primary RNA transcript.

Alternatively, the coding region utilized in the present invention may contain endogenous enhancers/promoters, splice junctions, intervening sequences, polyadenylation signals, etc. or a combination of both endogenous and exogenous control elements.

As used herein, the term “transcription unit” refers to the segment of DNA between the sites of initiation and termination of transcription and the regulatory elements necessary for the efficient initiation and termination. For example, a segment of DNA comprising an enhancer/promoter, a coding region and a termination and polyadenylation sequence comprises a transcription unit.

As used herein, the term “regulatory element” refers to a genetic element which controls some aspect of the expression of nucleic acid sequences. For example, a promoter is a regulatory element which facilitates the initiation of transcription of an operably linked coding region. Other regulatory elements are splicing signals, polyadenylation signals, termination signals, etc.

Transcriptional control signals in eukaryotes comprise “promoter” and “enhancer” elements. Promoters and enhancers consist of short arrays of DNA sequences that interact specifically with cellular proteins involved in transcription (Maniatis et al., Science 236:1237 [1987]). Promoter and enhancer elements have been isolated from a variety of eukaryotic sources including genes in yeast, insect and mammalian cells and viruses (analogous control elements, i.e., promoters, are also found in prokaryotes). The selection of a particular promoter and enhancer depends on what cell type is to be used to express the protein of interest. Some eukaryotic promoters and enhancers have a broad host range while others are functional in a limited subset of cell types (for review see Voss et al., Trends Biochem. Sci., 11:287 [1986]; and Maniatis et al., supra [1987]). For example, the SV40 early gene enhancer is very active in a wide variety of cell types from many mammalian species and has been widely used for the expression of proteins in mammalian cells (Dijkema et al., EMBO J., 4:761 [1985]). Two other examples of promoter/enhancer elements active in a broad range of mammalian cell types are those from the human elongation factor 1α gene (Uetsuki et al., J. Biol. Chem., 264:5791 [1989]; Kim et al., Gene 91:217 [1990]; and Mizushima and Nagata, Nucl. Acids. Res., 18:5322 [1990]) and the long terminal repeats of the Rous sarcoma virus (Gorman et al., Proc. Natl. Acad. Sci. USA 79:6777 [1982]) and the human cytomegalovirus (Boshart et al., Cell 41:521 [1985]).

As used herein, the term “promoter/enhancer” denotes a segment of DNA which contains sequences capable of providing both promoter and enhancer functions (i.e., the functions provided by a promoter element and an enhancer element, see above for a discussion of these functions). For example, the long terminal repeats (LTRs) of retroviruses contain both promoter and enhancer functions. The enhancer/promoter may be “endogenous” or “exogenous” or “heterologous.” An “endogenous” enhancer/promoter is one which is naturally linked with a given gene in the genome. An “exogenous” or “heterologous” enhancer/promoter is one which is placed in juxtaposition to a gene by means of genetic manipulation (i.e., molecular biological techniques) such that transcription of that gene is directed by the linked enhancer/promoter.

The presence of “splicing signals” on an expression vector often results in higher levels of expression of the recombinant transcript. Splicing signals mediate the removal of introns from the primary RNA transcript and consist of a splice donor and acceptor site (See e.g., Sambrook. et al., Molecular Cloning: A Laboratory Manual, 2nd ed., Cold Spring Harbor Laboratory Press, New York [1989], pp. 16.7-16.8). A commonly used splice donor and acceptor site is the splice junction from the 16S RNA of SV40.

Efficient expression of recombinant DNA sequences in eukaryotic cells requires expression of signals directing the efficient termination and polyadenylation of the resulting transcript. Transcription termination signals are generally found downstream of the polyadenylation signal and are a few hundred nucleotides in length. The term “poly A site” or “poly A sequence” as used herein denotes a DNA sequence which directs both the termination and polyadenylation of the nascent RNA transcript. Efficient polyadenylation of the recombinant transcript is desirable as transcripts lacking a poly A tail are unstable and are rapidly degraded. The poly A signal utilized in an expression vector may be “heterologous” or “endogenous.” An endogenous poly A signal is one that is found naturally at the 3′ end of the coding region of a given gene in the genome. A heterologous poly A signal is one which is one which is isolated from one gene and placed 3′ of another gene. A commonly used heterologous poly A signal is the SV40 poly A signal. The SV40 poly A signal is contained on a 237 bp Bam HI/Bcl I restriction fragment and directs both termination and polyadenylation (Sambrook, supra, at 16.6-16.7).

Eukaryotic expression vectors may also contain “viral replicons” or “viral origins of replication.” Viral replicons are viral DNA sequences which allow for the extrachromosomal replication of a vector in a host cell expressing the appropriate replication factors. Vectors which contain either the SV40 or polyoma virus origin of replication replicate to high copy number (up to 10⁴ copies/cell) in cells that express the appropriate viral antigen. Vectors which contain the replicons from bovine papillomavirus or Epstein-Barr virus replicate extrachromosomally at low copy number (˜100 copies/cell).

The term “stable transfection” or “stably transfected” refers to the introduction and integration of foreign DNA into the genome of the transfected cell. The term “stable transfectant” refers to a cell which has stably integrated foreign DNA into the genomic DNA.

As used herein, the term “stably maintained” refers to characteristics of recombinant (i.e., transgenic) animals that maintain at least one of their recombinant elements (i.e., the element that is desired) through multiple generations. For example, it is intended that the term encompass the characteristics of transgenic animals that are capable of passing the transgene to their offspring, such that the offspring are capable of maintaining the expression and/or transcription of the transgene. It is not intended that the term be limited to any particular organism or any specific recombinant element.

The terms “transient transfection” and “transiently transfected” refers to the introduction of foreign DNA into a cell where the foreign DNA fails to integrate into the genome of the transfected cell. The foreign DNA persists in the nucleus of the transfected cell for several days. During this time the foreign DNA is subject to the regulatory controls that govern the expression of endogenous genes in the chromosomes. The term “transient transfectanf” refers to cells which have taken up foreign DNA but have failed to integrate this DNA.

As used herein, the term “gene of interest” refers to the gene inserted into the polylinker of an expression vector. When the gene of interest encodes a gene which provides a therapeutic function, the gene of interest may be alternatively called a remedial gene.

As used herein, the terms “nucleic acid molecule encoding,” “DNA sequence encoding,” and “DNA encoding” refer to the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence.

As used herein, the term “adoptive transfer” is used in reference to the transfer of one function to another cell or organism.

Embryonal cells at various developmental stages can be used to introduce transgenes for the production of transgenic animals. Different methods are used depending on the stage of development of the embryonal cell. The zygote is the best target for micro-injection. In the mouse, the male pronucleus reaches the size of approximately 20 micrometers in diameter which allows reproducible injection of 1-2 picoliters (p1) of DNA solution. The use of zygotes as a target for gene transfer has a major advantage in that in most cases the injected DNA will be incorporated into the host genome before the first cleavage (Brinster et al., Proc. Natl. Acad. Sci. USA 82:4438-4442 [1985]). As a consequence, all cells of the transgenic non-human animal will carry the incorporated transgene. This will in general also be reflected in the efficient transmission of the transgene to offspring of the founder since 50% of the germ cells will harbor the transgene. Micro-injection of zygotes is the preferred method for incorporating transgenes in practicing the invention. U.S. Pat. No. 4,873,191 describes a method for the micro-injection of zygotes (the disclosure of this patent is hereby incorporated in its entirety).

The terms “overexpression” and “overexpressing” and grammatical equivalents, are used in reference to levels of mRNA to indicate a level of expression approximately 3-fold higher than that typically observed in a given tissue in a control or non-transgenic animal. Levels of mRNA are measured using any of a number of techniques known to those skilled in the art including, but not limited to Northern blot analysis. Appropriate controls are included on the Northern blot to control for differences in the amount of RNA loaded from each tissue analyzed.

The term “treatment” or grammatical equivalents encompasses the improvement and/or reversal of the symptoms associated with pathological TGF-β. “Improvement in the physiologic function” of the non-human transgenic animals of the present invention may be assessed using any of the measurements described herein, as well as any effect upon the transgenic animals' survival; the response of treated transgenic animals and untreated transgenic animals is compared using any of the assays described herein (in addition, treated and untreated non-transgenic animals may be included as controls). A compound which causes an improvement in any parameter associated pathological TGF-β when used in the screening methods of the instant invention may thereby be identified as a therapeutic compound.

The term “compound” refers to any chemical entity, pharmaceutical, drug, and the like that can be used to treat or prevent a disease, illness, sickness, or disorder of bodily function. Compounds comprise both known and potential therapeutic compounds. A compound can be determined to be therapeutic by screening using the screening methods of the present invention. A “known therapeutic compound” refers to a therapeutic compound that has been shown (e.g., through animal trials or prior experience with administration to humans) to be effective in such treatment. In other words, a known therapeutic compound is not limited to a compound efficacious in the treatment of symptoms associated with pathological TGF-β.

As used in the present invention, the term “transformation” refers to the introduction of foreign genetic material into a cell or organism. Transformation may be accomplished by any method known which permits the successful introduction of nucleic acids into cells and which results in the expression of the introduced nucleic acid. For example, transformation may be used to introduce cloned DNA encoding a normal or mutant receptor into a cell which normally does not express this receptor. “Transformation” includes but is not limited to such methods as transfection, microinjection, electroporation, and lipofection (liposome-mediated gene transfer). Transformation may be accomplished through use of any expression vector. For example, the use of baculovirus to introduce foreign nucleic acid into insect cells is contemplated. The term “transformation” also includes methods such as P-element mediated germline transformation of whole insects.

As used herein, the term “subject” refers to any animal (e.g., a mammal), including, but not limited to, humans, non-human primates, rodents, and the like, which is to be the recipient of a particular treatment. Typically, the terms “subject” and “patient” are used interchangeably herein in reference to a human subject.As used herein, the term “instructions for use for treating cancer in a subject” includes instructions for using the reagents for the treatment of cancer in a sample from a subject. In some embodiments, the instructions further comprise the statement of intended use required by the U.S. Food and Drug Administration (FDA) in labeling in vitro diagnostic products.

As used herein, the term “container” includes a receptacle, such as a carton, can, box, or jar, in which material is held or carried.

Experimental

The following examples are provided in order to demonstrate and further illustrate certain preferred embodiments and aspects of the present invention and are not to be construed as limiting the scope thereof.

In the experimental disclosure which follows, the following abbreviations apply: ° C. (degrees Centigrade); RT (room temperature); x g (times gravity); rpm (revolutions per minute); BSA (bovine serum albumin); IgG (immunoglobulin G); IM (intramuscular); IP (intraperitoneal); IV (intravenous or intravascular); SC (subcutaneous); H₂O (water); HCl (hydrochloric acid); aa (amino acid); bp (base pair); kb (kilobase pair); kD (kilodaltons); gm (grams); μg (micrograms); mg (milligrams); ng (nanograms); μl (microliters); ml (milliliters); mm (millimeters); nm (nanometers); μm (micrometer); M (molar); mM (millimolar); μM (micromolar); U (units); V (volts); MW (molecular weight); sec (seconds); min(s) (minute/minutes); hr(s) (hour/hours); MgCl₂ (magnesium chloride); NaCl (sodium chloride); EGF (epidermal growth factor); AEBSF (4-(2-aminoethyl)-benzene sulfonyl fluoride); OD₂₈₀ (optical density at 280 nm); OD₆₀₀ (optical density at 600 nm); PAGE (polyacrylamide gel electrophoresis); PBS (phosphate buffered saline [150 mM NaCl, 10 mM sodium phosphate buffer, pH 7.2]); PCR (polymerase chain reaction); PEG (polyethylene glycol); PMSF (phenylmethylsulfonyl fluoride); RT-PCR (reverse transcription PCR); SDS (sodium dodecyl sulfate); Tris (tris(hydroxymethyl) aminomethane); w/v (weight to volume); v/v (volume to volume); ATCC (American Type Culture Collection, Rockville, Md.); R&D Systems (R&D Systems, Inc., Minneapolis, Minn.); Life Technologies (Life Technologies, Inc., Gaithersburg, Md.); Invitrogen (Invitrogen Corp., Carlsbad, Calif.); Kodak (Eastman Kodak Co., New Haven, Conn.); Boehringer Mannheim (Boehringer Mannheim, Ind.polis, Ind.); Jackson (Jackson ImmunoResearch Laboratories, Inc., West Grove, Pa.); Pierce (Pierce Chemical Co., Rockford, Ill.); Anilytics (Anilytics Inc., Gaithersburg Md.); BioFX (BioFX, Owings Mills, Md.); and Sigma (Sigma Chemical Co., St. Louis, Mo.).

### EXAMPLE 1

### Construction of the MMTV-SR2F Transgenic Mouse

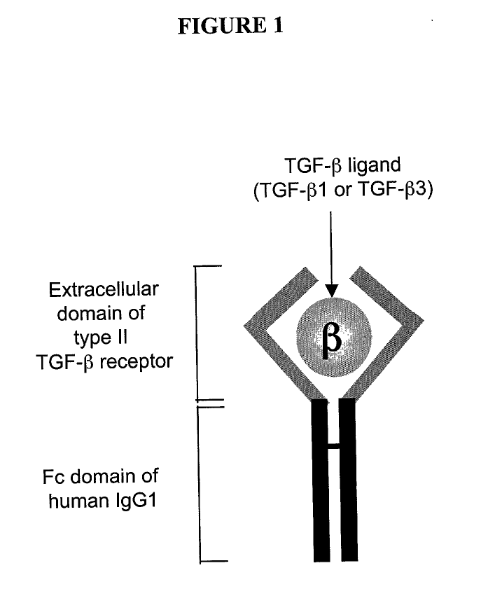

In this Example, experiments conducted to generate a mouse that expresses high levels of a soluble TGF-β antagonist are described. The soluble antagonist used is of the “receptor-body” class and consists of the extracellular ligand binding domain of the type II TGF-β receptor fused to the Fc domain of human IgG,. FIG. 1 provides a schematic diagram of this antagonist referred to as “SR2F” for “soluble type II TGF-β receptor-Fc fusion protein” (Komesli et al., Eur. J. Biochem., 254:505-513 [1998]).

This antagonist binds TGF-β1 and TGF-β3 with high affinity, but does not bind TGF-β2 (See, Komesli et al., supra; and Tsang et al., Cytokine 7:389-397 [1995]). The mouse mammary tumor virus long terminal repeat (MMTV-LTR) promoter/enhancer element was chosen to drive transgene expression. This promoter expresses primarily in the mammary gland, beginning at puberty. It was chosen for two reasons: (i) to drive high level of expression in the mammary gland so the impact of TGF-β on mammary tumorigenesis could be assessed; and (ii) to avoid high level expression in the embryo or neonate, since TGF-β1 and TGF-β3 null mice show embryonic or perinatal lethality (Bonyadi et al., Nat. Genet., 15:207-211 [1997]; and Kaartinen et al., Nat. Genet., 11:415-421 [1995]).

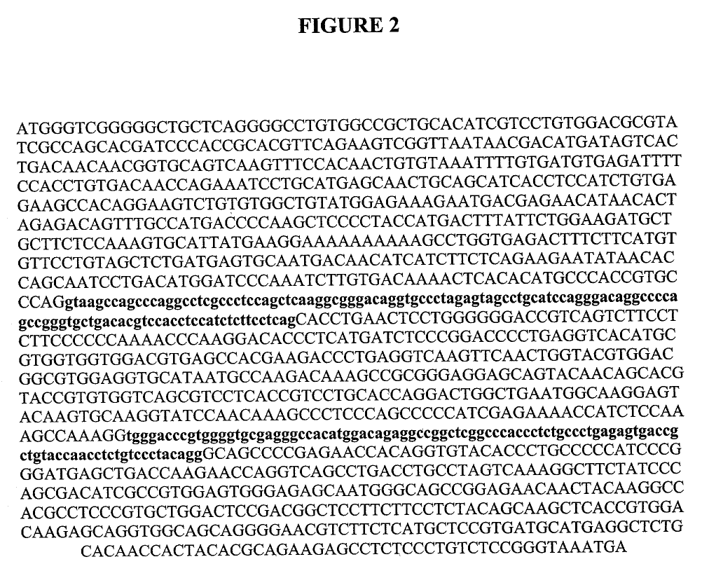

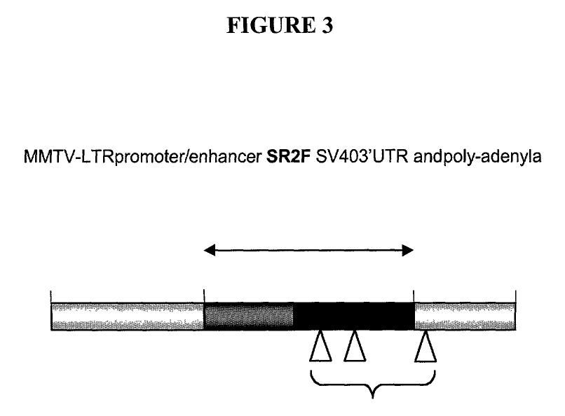

A. Transgene Design and Validation First, generation of SR2F expression and transgene constructs is described. Plasmid JP109-6 encoding the SR2F was obtained through a collaboration with Dr. Monica Tsang (R&D Systems, Inc., Minneapolis, Minn.). The sequence included two small introns in the Fc domain (See, FIG. 2), since introns generally increase expression of transgenes in vivo. EcoRI linkers were added to the SR2F insert for subcloning into the EcoRI site of the pSKMMTV-SVPA vector (gift of Dr. Phil Leder, Harvard Medical School) in order to generate the transgene. Excision with BglII and SpeI produced a transgene in which the SR2F coding sequence is flanked by the MMTV-LTR promoter/enhancer and SV40 3′UTR and polyadenylation signal (See, FIG. 3). The SV40 3′UTR also contains a small intron. For in vitro validation, the SR2F was further subcloned into the HindIII/XbaI sites of the pcDNA3 mammalian expression vector (Invitrogen Corp., Carlsbad, Calif.), as the CMV promoter in the pcDNA3 vector drives much higher level expression in transfected cells than does the MMTV-LTR promoter/enhancer.

B. Stable Transfectants

The human breast cancer cell line MDA MB435 (gift of Dr. Stephen Byers, Lombardi Institute, Georgetown University, Washington D.C.) was stably transfected with pcDNA3 vector alone, pcDNA3-SR2F and pcDNA3-DNR, where DNR is a membrane-bound dominant negative form of the type II TGF-β receptor (with a deletion of the entire intracellular domain). The DNR served as a positive control for the ability to block the biological activity of added TGF-β. Clones with high level expression of SR2F or DNR mRNA were selected for further study.

C. Growth Inhibition Assays

Transfected cells were seeded at 10⁵ cells/well in 24 well-plates in DMEM, 10% FBS and 1% penicillin-streptomycin. After growing for 24 hours, cells were switched to assay medium containing DMEM, 0.1% FBS, 10 ng/ml EGF, 1% penicillin/streptomycin, with or without the addition of 5 ng/ml TGF-β1 (R&D Systems). After a further 22 h, cells were pulsed with ³H-thymidine (“³H-Thy”) for 2 hours and then harvested to determine the extent of incorporation of ³H-Thy into DNA. For determining the molar ratio of SR2F required to neutralize a given amount of TGF-β, the highly sensitive MvlLu mink lung epithelial cell line was used (American Type Culture Collection, Rockville, Md.). The assay format was as for the MDA MB435 cells, except that 2 pM TGF-β1 was used, with or without added purified SR2F protein (R&D Systems, Inc., Minneapolis, Minn.) over a concentration range of 0 to 200 pM.

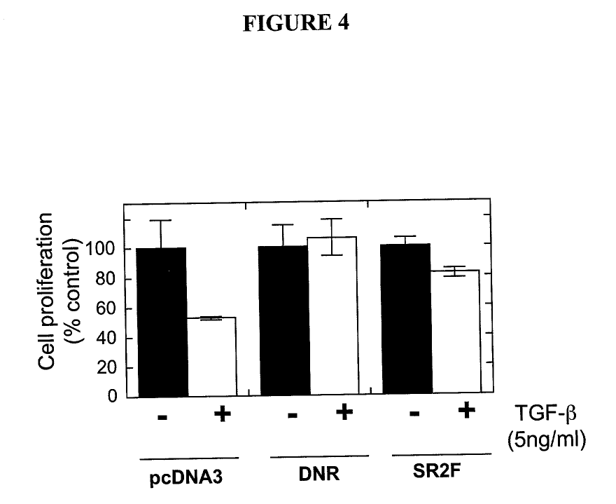

MDA MB435 cells stably expressing the SR2F were partially resistant to growth inhibition by TGF-β1, while cells expressing the DNR were fully resistant, as shown in FIG. 4. This indicates that the SR2F antagonist construct is biologically active. The lower efficiency of the SR2F construct in vitro when compared with the membrane bound DNR probably reflects dilution of the secreted SR2F into the relatively large volume of the cell growth medium.

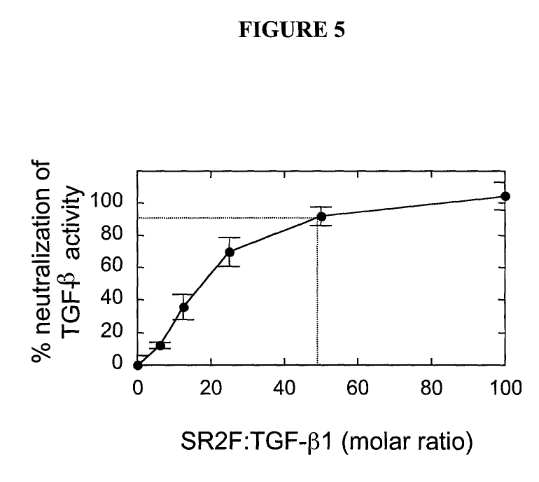

The molar ratio of SR2F required to neutralize TGF-β activity was determined in a growth inhibition assay using a more sensitive indicator cell line. As indicated in FIG. 5, 90% reversal of the biological activity of TGF-β was activated at a molar ratio of SR2F: TGF-β1 of 50:1.

D. Establishment of the Transgenic Mouse Line

A linear 4.3 Kb BglII-SpeI fragment containing the SR2F transgene was excised from the parental plasmid, and injected into the pronuclei of inbred FVB/N zygotes using methods known in the art previously described (See e.g., Hogan et al., Manipulating the Mouse Embryo, supra). Six founders were obtained, of which 5 showed germline transmission. Offspring were screened for transgene expression by Northern blot and using a specific ELISA assay to detect SR2F protein. Of the two expressing lines, one line (MMTV-SR2F-G22M) showed 100-fold higher expression of SR2F protein in the mammary gland than the other, and was used for all further analyses. This line had a single integrated copy of the transgene. Circulating SR2F levels in transgenic mice were generally maintained at >200 ng/ml throughout the lifespan of the animal, due to endogenous synthesis.

E. Northern Blot and in situ Hybridization Analysis

For Northern blot analysis, total RNA was extracted from tissues using the Trizol reagent, according to the manufacturer's instructions (Life Technologies, Inc., Gaithersburg, Md.). Then, 15 μg of RNA was separated on a 1% agarose/formaldehyde gel, transferred to Nytran and hybridized with a 520 bp ³²P labeled probe specific for the Fc domain of SR2F. In situ hybridization was performed on 5 μm paraformaldehyde-fixed tissue sections. The 520 bp probe specific for the Fc domain was subcloned into the pCR2.1 vector (Invitrogen Corp., Carlsbad, Calif.) and the plasmid was linearized with BamH1 or HindIII and transcribed using the Boehringer Mannheim Genius Kit 4 (Boehringer Mannheim) to generate sense and antisense probes. Hybridization of slides using digoxygenin-labeled probes was performed as known in the art (See e.g., Jakowlew et al., Mol. Carcinogen., 22:46-56 [1998]). Slides were counterstained with Nuclear Fast Red.

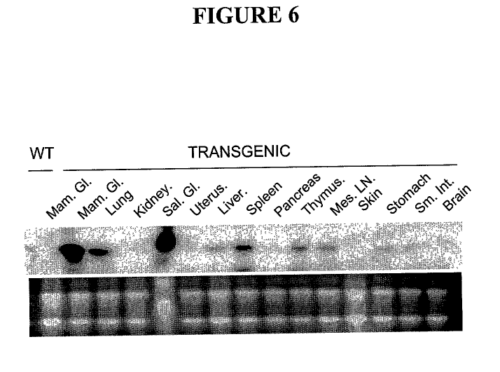

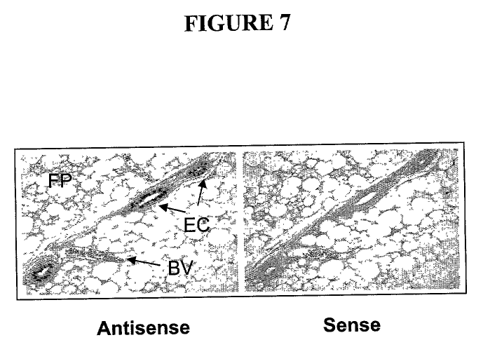

Northern blot analysis of tissues from a 4 month-old homozygous virgin female transgenic mouse showed high level expression of SR2F mRNA in the mammary gland and salivary gland, with lower level expression in the lung, spleen, thymus and lymph nodes, as indicated in FIG. 6. This expression pattern is characteristic of the MMTV-LTR. In situ hybridization analysis showed that in the mammary gland, transcription of the transgene was restricted to the mammary epithelial cells, and was not seen in the cells of the fat pad or blood vessels tested, as shown in FIG. 7.

F. ELISA Assay for SR2F

A highly sensitive and specific ELISA assay was developed for the quantitation of SR2F. Tissue extracts were prepared by homogenizing tissues with extraction buffer (1% Nonidet P40, 150 mM NaCl, 50 mM Tris HCI pH 7.4, and 20 μg/ml 4-(2-aminoethyl)-benzene sulfonyl fluoride (AEBSF)) using 2 ml buffer/100 mg tissue, and clarifying extracts by centrifugation at 10,000× g for 20 min. Nunc Maxisorp microtitre ELISA plates (Nalge Nunc International, Rochester, N.Y.) were coated overnight at 4° C. with 1 μg/well of goat anti-human IgG (#109-005-098) (Jackson ImmunoResearch Laboratories, West Grove, Pa.). After washing twice with wash buffer (“WB”: 2 mM imidazole-buffered saline, 0.02% Tween 20), wells were blocked for 1 h at RT with TBS/casein (BioFX Laboratories Inc., Owings Mills, Md.) and washed again. Samples or purified SR2F standard (1-100 pg/well) serially diluted in TBS/casein were added and incubated for 1 h at RT. After washing 3 times, wells were incubated with 12.4 ng/well of biotinylated anti-TGF-β receptor type II (#BAF241) (R&D Systems, Inc., Minneapolis, Minn.) for 1.5 h at RT. After a further 3 washes, wells were incubated with a 1 :10,000 dilution of streptavidin-conjugated peroxidase (#016-030-084) (Jackson ImmunoResearch Laboratories, West Grove, Pa.) for 1 h at RT. Wells were washed 4x with wash buffer and once with deionized water. Peroxidase substrate (# TMBW-0100-01) (BioFX Laboratories Inc., Owings Mills, Md) was added to each well and the color allowed to develop for 30 minutes before stopping the reaction with 0.1 ml/well of 1 N HCl and reading the OD₄₅₀ₙₘ.

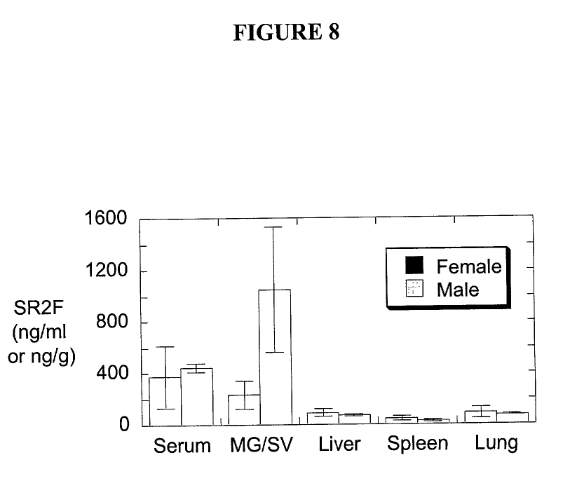

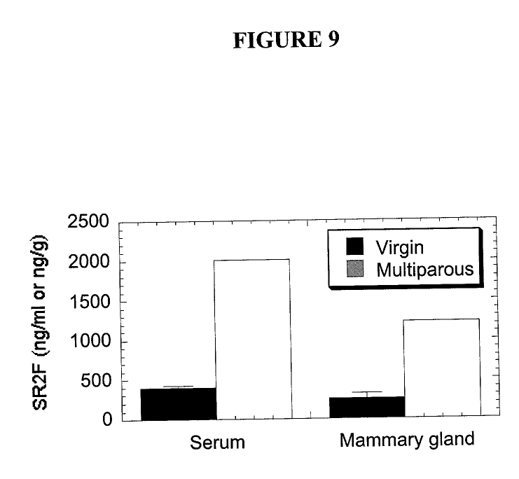

Levels of SR2F in different tissues were quantitated using the ELISA assay, as shown in FIG. 8. In 2.5 month old homozygous virgin mice, highest SR2F expression was seen in the mammary gland of females (˜230 ng/g tissue) and the seminal vesicle of males (˜1000 ng/g). Both sexes had circulating levels of SR2F of ˜400 ng/ml. These levels varied somewhat from litter to litter, and serum levels tended to decrease with age, leveling off at 150-300 ng/ml. The very high levels of circulating SR2F in young mice are due to the additional presence of maternally transferred SR2F, as discussed herein. All other tissues, except the brain, had low but detectable levels of SR2F (range 30-150 ng/g; not shown). The presence of SR2F protein in organs, such as the kidney, which show no SR2F RNA, probably reflects sequestration of SR2F from the circulation. Circulating and tissue levels of SR2F were considerably increased (up to 5-fold) in parous females, as shown in FIG. 9, due to enhanced expression from the MMTV-LTR during pregnancy and lactation.

G. Western Blot Analysis

Mammary glands from transgenic and wild-type mice were extracted by homogenization in 3 ml of ice-cold lysis buffer (1% NP-40, 150 mM NaCl, 50 mM Tris pH 8.0, 75 μg/ml AEBSF) per gram of tissue. Extracts were clarified by 2 rounds of centrifugation at 15,000× g for 20 min. at 4° C. The protein concentration in the extract was determined using the BCA Protein Assay kit according to manufacturer's instructions (Pierce Chemical Co., Rockford, Ill. ). Then, 20-50 μg/well extracted protein or 0.2-0.5 ng of purified SR2F was electrophoresed on a 4-12% Tris/glycine gel under non-reducing conditions, and blotted onto nitrocellulose membranes. Blots were probed with either Biotin-SP-conjugated AffiniPure Goat Anti-Human IgG, Fc Fragment specific antibody (#109-065-098) (Jackson ImmunoResearch Laboratories, West Grove, Pa.) at a final concentration of 0.2 μg/ml. Blots were developed using streptavidin-peroxidase and the SuperSignal West Pico detection system (Pierce Chemical Co., Rockford, Ill.). A replicate blot was probed with primary antibody that had been pre-blocked for 4 hours at 4° C. with a 25-fold excess of human immunoglobulin G (ChromPure Human IgG, #001-000-003, Jackson ImmunoResearch Laboratories, West Grove, Pa.). ).

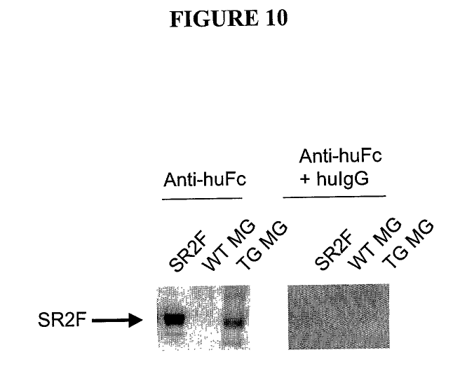

Western blot analysis of mammary gland extracts showed a specific band of 130 kDa under non-reducing conditions in the transgenic, but not the wild-type mammary glands. It is the same size as the purified SR2F, indicating that the SR2F is being correctly dimerized and glycosylated in the transgenic mammary gland, as indicated in FIG. 10.

H. Ligand Affinity Crosslinking

Sera from 3-month old virgin transgenic or wild-type mice were diluted 16-fold in PBS and used for cross-linking. Purified SR2F (2 ng/0.1 ml) was spiked into diluted wild-type serum or phosphate buffered saline (PBS) as a positive control. ¹²⁵I-TGF-β1 (131 μCi/μg) was added to a final concentration of 0.4 nM and the reaction mix was incubated on ice for 1-5 h. The bifunctional cross-linking reagent disuccinimidylsuberate (Pierce Chemical Co., Rockford, Ill.) was added to a final concentration of 1 mM and incubated for 30 min at RT before quenching the reaction with 1/20 vol. 1 M Tris, pH 7.5. Samples were run on a 6% Tris-glycine gel under non-reducing conditions, and the gel was dried and exposed to film for 1 day.

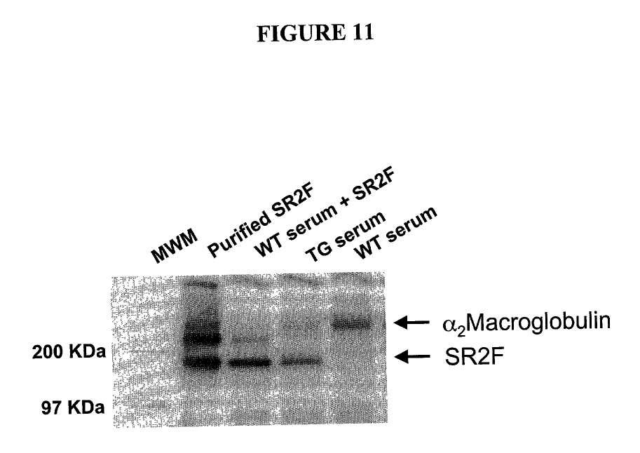

Thus, the ability of the SR2F made in vivo to bind to TGF-β was determined using a ligand affinity cross-linking assay as described above. ¹²⁵I-TGF-βl bound to an identical band of 155 kDa in transgenic serum and wild-type serum spiked with purified SR2F (See, FIG. 11). In contrast, TGF-β bound only to a high molecular weight band corresponding to α2 macroglobulin in wild-type serum. This indicates that the SR2F made in vivo is correctly folded and capable of binding TGF-β with a much higher affinity than the major serum binding protein α2 macroglobulin, which is present in great excess.

I. Determination of in vivo Half-Life of SR2F

A hemizygous transgenic female was bred to a wild-type male mouse. Offspring were genotyped at weaning. Blood was serially drawn from the saphenous vein of wild-type offspring at 5, 8, 12 and 20 weeks of age. Serum was prepared and SR2F levels were determined using the ELISA assay. The in vivo half-life was calculated from the decay curve.

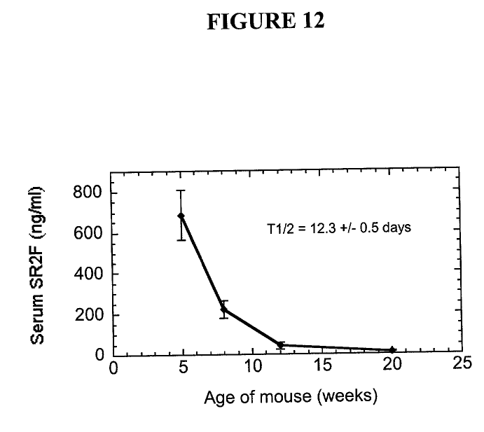

Upon breeding of transgenic mice through hemizygous/wild-type matings, detectable SR2F was detected in wild-type offspring when the mother was transgenic. This was not seen with transgenic fathers, and indicated that the SR2F is maternally transferred, either transplacentally and/or in the milk. This allowed the determination of the half-life of SR2F in vivo by following the time-course of decay of maternally transferred SR2F in the wild-type offspring of hemizygous transgenic mothers, as shown in FIG. 12. Based on the data obtained, SR2F has an in vivo half-life of 12.3+/−0.5 days.

### EXAMPLE 2

### The MMTV-SR2F Transgenic Mouse is Protected Against Metastasis

As indicated above, TGF-βs are overexpressed in many advanced tumors, particularly at the leading invasive edge of the tumor. Metastases frequently show higher level of TGF-β expression than do the parental tumors, and metastatic cell lines can activate more latent TGF-β than can their non-metastatic counterparts. Pretreatment of cell lines in vitro with TGF-β enhances their metastatic ability, while neutralizing antibodies to TGF-β inhibit metastasis. All these data point to a positive role for TGF-β in the promotion of metastasis. Since the transgenic mouse model of the present invention has high circulating levels of a TGF-β antagonist, experiments were conducted in order to determine whether the mice were protected against the development of metastases. In initial studies, the hypothesis was tested using a short-term tail-vein injection assay of experimental metastasis. Isogeneic metastatic melanoma cells were injected into the tail vein of experimental and control mice and scored for metastases in distant organs. In these experiments, melanoma cells were used because an isogeneic metastatic breast cancer cell line was not available for use.

A. Suppression of Metastasis in a Tail Vein Metastasis Model

These studies used the isogeneic amelanotic melanoma cell line 37-32 that was derived from a melanoma arising in an FVB/N mouse transgenically overexpressing hepatocyte growth factor from the metallothionein promoter (HGF/SF transgenic mouse line MH-37) (See, Otsuka et al., Canc. Res., 58:5157-5167 [1998]). These cells have been previously characterized as being metastatic in a tail vein injection assay (Otsuka et al., Mol. Cell Biol., 20:2055-2065 [2000]). Cells were cultured in growth medium comprising DMEM, 10% FBS, 5 μg/ml insulin, and 5 ng/ml EGF. Exponentially growing cells were harvested by trypsinization and centrifugation, and resuspended in normal growth medium without calcium or magnesium to a concentration of 10⁷ cells/ml. Transgenic and age- and sex-matched wild-type mice were each injected with 10⁶ cells in 0.1 ml of medium intravenously via the tail vein using a 26 gauge needle. For the pilot study, 5 transgenic and 4 wild-type male mice aged 7 weeks were used, and mice were euthanized 5 weeks after injection with cells. For the larger scale study, 3-4 month old male mice were used. Five mice per genotype group were euthanized 3 weeks after injection with cells, and 10 mice each per genotype group were euthanized 4 and 5 weeks after injection. Mice were examined grossly at necropsy for the presence of metastases in internal organs. Organs were fixed in neutral buffered formalin, paraffin-embedded, and 5 μm sections were stained with hematoxylin and cosin (H&E). Sections were examined for histological evidence of metastases, and the number of metastases/organ were counted by a board-certified Veterinary Pathologist. For the liver, 2 sections of each lobe were analyzed, while for the remaining organs a longitudinal cross section was used. Blood was collected at the time of necropsy for determination of SR2F levels in the serum.

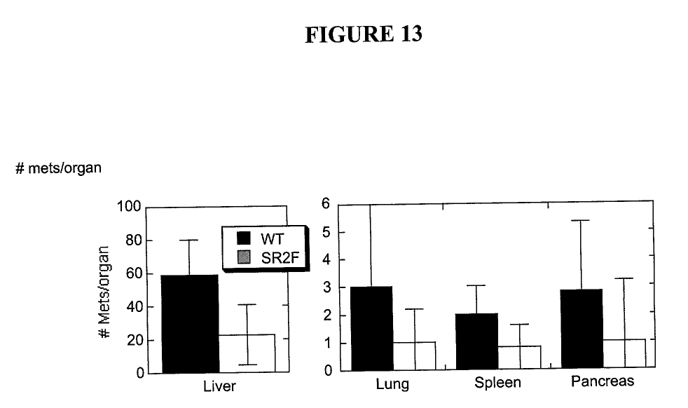

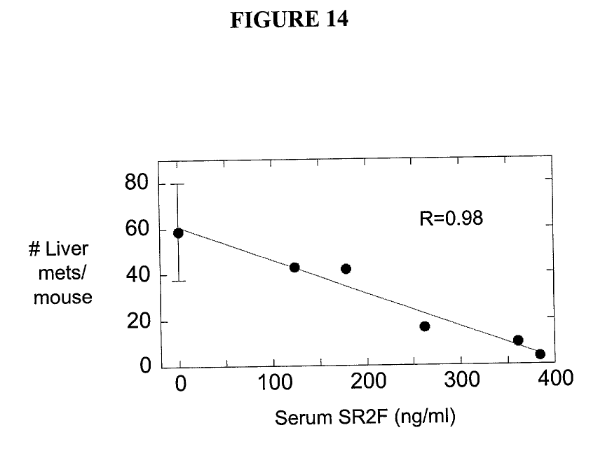

In the pilot study, metastases were seen on gross examination in the liver, lung, spleen and pancreas. A three-fold decrease in the number of histologically confirmed metastases/mouse was seen for all organs examined in transgenic mice as compared to wild-type mice, as shown in FIG. 13. In the liver, which was the site of the largest number of metastases, this reduction was statistically significant (p=0.03; 2-tailed t-test). Serum levels of SR2F were quite variable in these young mice due to maternal transfer. The number of metastases/liver decreased linearly with increasing levels of circulating SR2F (See, FIG. 14) with a correlation coefficient of 0.98. Linear extrapolation suggested that metastasis would be totally eliminated at a circulating SR2F level of 420 ng/ml, sufficient to neutralize 100 pM TGF-β.

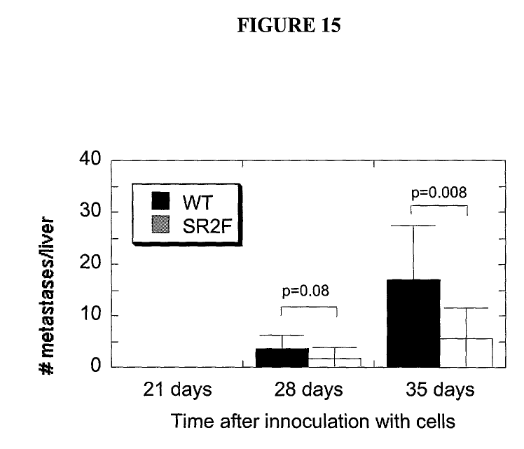

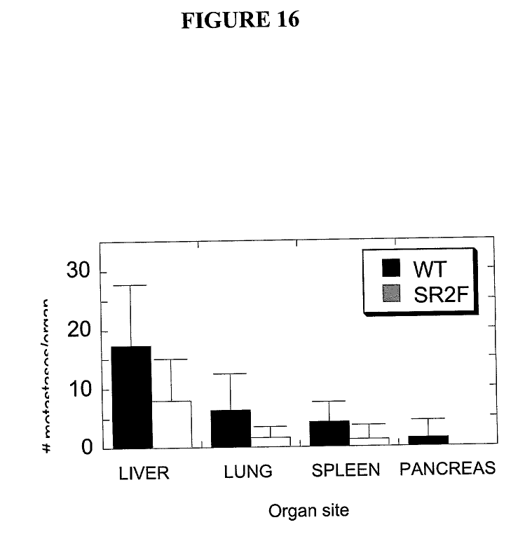

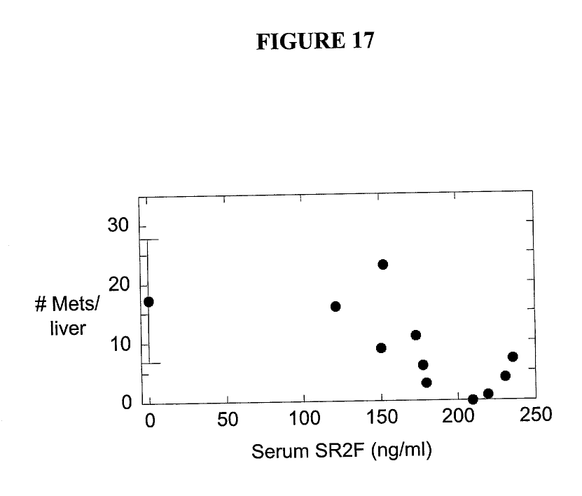

In the larger-scale study, grossly visible metastases were already evident in the liver by 28 days after injection with the cells, and by 35 days the number of visible metastases had increased ˜4-fold. At both time points, there were ˜3× more metastases/liver in the wild-type mice when compared with the transgenic SR2F mice, as indicated in FIG. 15. The overall number of metastases/organ were lower in this experiment than in the pilot experiment. However, the SR2F mice consistently showed 2-3× fewer histologically confirmed metastases/organ than did the wild-type mice (See, FIG. 16). For the liver, which had the greatest number of metastases, this difference was statistically significant (p=0.03, 2-tailed t test). There was less of a spread of serum SR2F levels in this experiment because the mice were older, but again, lower numbers of metastases were observed in mice with higher levels of circulating SR2F (See, FIG. 17). All metastases were histologically confirmed.

B. Suppression of Metastasis from an Autochthonous Mammary Tumor Model System

Since the soluble antagonist was effective in protecting against metastasis in a tail-vein assay, the determination was then made as to whether it was effective at preventing metastasis from a primary tumor arising in its natural site. In addition, since TGF-β is thought to act as a tumor suppressor in the early stages of tumorigenesis, determination of whether there were any adverse early tumor promoting effects of chronic exposure to the TGF-β antagonist during all stages of tumorigenesis was made. The MMTV-neu transgenic mouse model of mammary tumorigenesis gives rise to palpable mammary tumors with a median time of onset of ˜34 weeks, with up to 70% of the mice showing metastasis to the lungs (See, Proc. Natl. Acad. Sci. USA, 89:10578-10582 [1992]). In this model, mammary tumorigenesis is initiated by overexpression of the rat homolog of the HER2/erbB2/neu protooncogene. The MMTV-SR2F mice were crossed with the MMTV-neu mice to look at the impact of TGF-β antagonist expression on development of the primary mammary tumors and their subsequent metastatic behavior as well as to determine whether there were any adverse tumor promoting effects following chronic exposure to the TGF-β antagonist during all stages of tumorigenesis.

Homozygous MMTV-neu mice (The Jackson Laboratory, Bar Harbor, Me.) were crossed with homozygous MMTV-SR2F mice or with FVB/N control mice to generate two experimental cohorts. Group I had the Neu oncogene alone (Neu). Group II was bi-transgenic, having both Neu and the SR2F antagonist (Neu/SR2F). Both transgenes were hemizygous. Female mice were cycled through one round of pregnancy in order to increase expression of the Neu and SR2F transgenes from the hormonally-responsive MMTV promoter/enhancer. Group I contained 29 mice, and group II contained 38 mice. Mice were monitored for tumor development by biweekly palpation of the mammary glands. Mice were euthanized when any primary tumor reached 2 cm in diameter, or if the mouse appeared morbid. Tumor volume was calculated by the formula V=LS×0.4 where L and S are the longest and shortest dimensions, respectively. (See, Fueyo et al., Nat. Med., 4:685-690 [1998]). At the time of euthanasia, mammary glands and lungs were harvested for histological analysis, and serum was collected for assay of circulating SR2F levels. H&E stained sections of mammary glands and lungs were examined by a Board Certified Veterinary Pathologist for the presence of primary mammary tumors and of lung metastases.

In the 62 week time frame of the study, 25/29 (86.2%) of the mice in the Neu group developed palpable mammary tumors and 33/38 (86.8%) of the mice in the Neu/SR2F group did. Tumor-bearing mice were necropsied for further evaluation when a primary tumor reached 2 cm diameter or the mouse showed signs of morbidity. At the time of euthanasia, circulating SR2F levels in tumor-bearing mice were 18.0+/−10.2 μg/ml (range 4.3 to 38.8 μg/ml serum). This is substantially higher than SR2F levels normally found in parous SR2F mice (˜1 μg/ml), and is contemplated as reflecting production of SR2F by cells of the tumor.

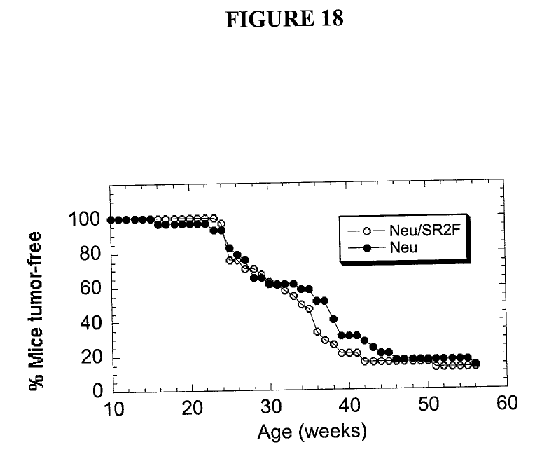

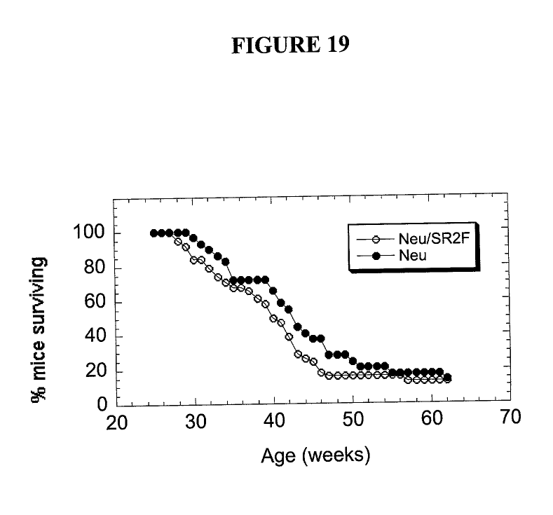

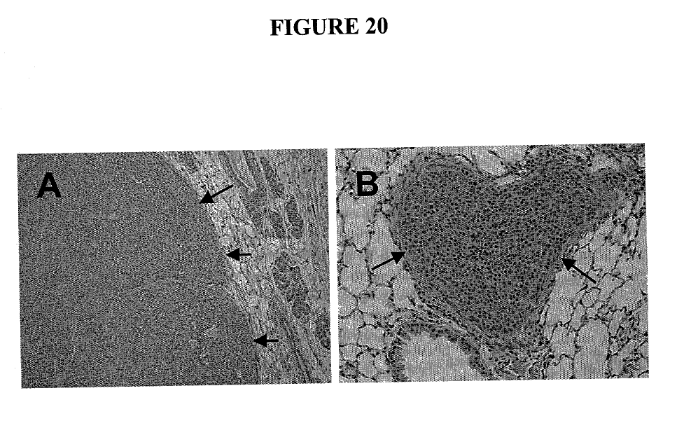

Tumor latency, as determined by the age at which a palpable tumor was first detected, was unaffected by the presence of SR2F, as indicated in FIG. 18. Similarly, as indicated in Table 1, tumor multiplicity and total tumor burden/mouse were unaffected by the presence of SR2F. Neu mice had 2.4+/−1.8 tumors/mouse, while neu/SR2F mice had 2.4+/−1.7 tumors/mouse, and the average total tumor burden for the neu mouse was 1.8+/−1.0 cm³, while for the neu/SR2F mouse it was 1.7+/−1.0 cm³. In addition, there was no statistically significant effect of SR2F on survival, as shown in FIG. 19. It should be noted, that in this metastatic breast cancer model, “survival” is determined primarily by the size of the primary tumor, and not by the presence or absence of metastases. This is because ACUC guidelines require mice to be euthanized when the primary tumor reaches 2 cm in diameter, regardless of the apparent state of health of the mouse. The survival curves are not statistically different (p=0.3, Log-rank test). The histology of a representative primary tumor and lung metastasis from a neu mouse are shown in FIG. 20. The histopathology was not affected by the presence of SR2F (not shown).

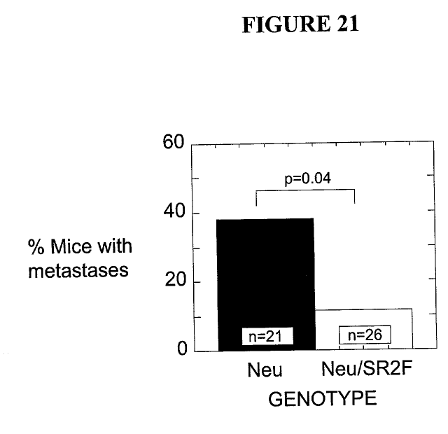

In contrast to the lack of effects on tumor latency, multiplicity, and size of the primary mammary tumors, the presence of SR2F significantly decreased the incidence of lung metastases (See, FIG. 21). It is contemplated that prolonged exposure to SR2F can protect the mouse against metastasis from an endogenous primary tumor, without accelerating formation of the primary tumor. The neu/SR2F bigenic mice showed a 3.3× decrease in the number of mice with lung metastases when compared with the neu mice (p=0.04; 2-tailed Fisher exact probability test). However, six mice in the neu/SR2F group and two mice in the neu group were not evaluable for metastasis as the lungs did not inflate properly at necropsy.

TABLE 1
Effect of SR2F on Tumor Multiplicity
and Cumulative Tumor Burden
in the MMTV-neu Mammary Tumor Model

Genotype

| | Neu | Neu/SR2F |
|---|---|---|
| Tumor Multiplicity | 2.4 ± 1.8 | 2.4 ± 1.7 |
| (# Tumors/Mouse) | | |
| Tumor Burden (cm) | 1.8 ± 1.0 | 1.7 ± 1.0 |

### EXAMPLE 3

### Absence of Phenotype in Unchallenged MMTV-SR2F Mice

The conceptual problem with the long-term use of a systemic TGF-β antagonist for treatment or prevention of TGF-β-induced pathologies has always been that there would likely be many undesirable side-effects due to the neutralization of endogenous TGF-βs in normal tissues. In particular, loss of TGF-β function is associated with aberrant proliferation in normal epithelia, increased tumorigenesis and immune system dysfunction. TGF-β1 null mice have profound immune system defects, and develop a lethal multifocal inflammatory syndrome with many features of autoimmune disease. In addition, they develop colon cancer with high incidence. Cohorts of age-matched SR2F and wild-type FVB/N mice were analyzed for evidence of pathology induced by prolonged exposure to the SR2F.

A. Necropsy Data

Complete necropsies were performed on 22 female FVB/N mice and 20 female SR2F+/+ mice, aged 16-26 months, with a mean age of 20+/−2 months for the FVB/N group and 21+/−4 months for the SR2F group. The SRF+/+ group included 8 parous mice (40% of total), while the FVB/N control group also included 5 parous mice (23% of total). All other mice were virgins. At necropsy all mice were examined for the presence of gross lesions or abnormalities. In these analyses, 33 organs were routinely harvested for histologic observation, and any tissues additionally showing abnormalities on gross necropsy were also harvested for microscopic examination. H&E stained sections of formalin-fixed paraffin-embedded tissue from each mouse were examined by a board-certified veterinary pathologist (Dr. Miriam Anver, DVM, PhD) for evidence of pathology. Serum and select mammary glands were harvested at the time of necropsy for determination of SR2F levels by ELISA assay.

At the time of necropsy, serum SR2F levels in the transgenic group were 930+/−551 ng/ml (range 207-1958 ng/ml), while the SR2F levels in the mammary gland were 496+/−287 ng/g (range 127-976 ng/g). The mean circulating serum SR2F levels of 930 ng/ml would be sufficient to neutralize 5 ng/ml (200 pM) of TGF-β (See, FIG. 5). This level of TGF-β is more than saturating for most known biological responses. Thus, it is contemplated that the amount of SR2F in these aged mice is adequate for neutralization of endogenous TGF-β.

There was no increase in the incidence of neoplasms in any organs in the aged SR2F mice when compared with FVB/N controls, as indicated in Tables 2A and 2B, below. In particular, there was no increased incidence of spontaneous mammary tumors, as is seen in mice with decreased TGF-β receptor function in the mammary gland (Gorska et al., supra), and no increase in colon tumors as is seen in the TGF-β1 null mouse (Engle et al., supra). Taking into consideration all neoplasms, in the FVB/N group, there were a total of 36 neoplasms in 22 mice (mean 1.6+/−1.4 neoplasms/mouse), while in the SR2F group there were a total of 29 neoplasms in 20 mice (mean 1.5=/−1.1 neoplasms/mouse). In Table 2B, the incidence data is broken down by organ site. The incidence is given as the number of mice with the particular tumor divided by the number of mice examined for that organ. Not all mice were examined for all organs.

Statistically significant increases in the incidence of chronic cardiomyopathy, pancreatic islet cell hyperplasia, eosinophilic degeneration of the spinal cord, lymphoid hyperplasia of the thymic medulla and lymphocytic infiltrates were seen in aged SR2F mice when compared with aged FVB/N mice, as shown in Table 3B, below. For these Tables, not all mice were evaluated for all organs. However, the number of mice examined is given for each organ. Severity of the lesions was assessed on a 4-step grading scale of minimal, mild, moderate, and marked.

Statistical analysis was done using the Fisher Exact T-test. Where present, the increase in lymphocytic infiltrates was in the minimal to mild severity range and there was no evidence for the diffuse vasculitis and severe multifocal inflammation observed in the TGF-β1 knockout mouse (Shull et al., supra; and Kulkarni et al., Proc. Natl. Acad. Sci. USA 90:770-774 [1993]). Similarly the increased incidence of chronic cardiomyopathy (an age-related degenerative lesion of mice with no direct counterpart in humans) was in the minimal to mild severity range and there was no evidence for the severe myocarditis that is a prominent feature of the TGF-β1 null mouse (Kulkarni et al., supra). The other non-neoplastic pathologies, where present, were also not severe, and did not appear to be associated with any morbidity. SR2F mice lived as long as wild-type controls.

TABLE 2A
Microscopic neoplastic findings summary

| | FVB/N mice | MMTV-SR2F mice |
|---|---|---|
| Total # primary tumors/group | 36 | 29 |
| Total # mice/group | 22 | 20 |
| Total # animals with tumors | 17/22 (77%) | 16/20 (80%) |
| Animals with multiple tumors | 9/22 (41%) | 9/20 (45%) |
| Total benign tumors | 21/36 (58%) | 20/29 (69%) |
| Total malignant tumors | 15/36 (42%) | 9/29 (31%) |
| Total malignant with metastasis | 4/36 (11%) | 2/29 (7%) |
| Mean tumors/mouse | 1.6+/−1.4 | 1.5+/−1.1 |

TABLE 2B
Incidence of neoplastic microscopic
findings in aged FVB/N and MMTV-
SR2F mice, listed by tumor site.

| | FVB/N | | SR2F | |
|---|---|---|---|---|
| TUMOR TYPE | Incidence | % | Incidence | % |
| Gall bladder, papilloma | 1/16 | 6.3 | 1/19 | 5.3 |
| Harderian gland, adenoma | 1/18 | 5.6 | 1/20 | 5.0 |
| Hematopoietic neoplasm, | 3/18 | 16.7 | 1/19 | 5.3 |
| histiocytic sarcoma | | | | |
| Hematopoietic neoplasm, | 0/18 | 0 | 1/19 | 5.3 |
| lymphoma FCC | | | | |
| Kidney, lipoma | 0/18 | 0 | 1/20 | 5.0 |
| Liver, hepatocellular adenoma | 1/18 | 5.6 | 0/20 | 0 |
| Lung, alveolar adenoma | 8/17 | 47.1 | 5/20 | 25.0 |
| Lung, alveolar carcinoma | 4/17 | 23.5 | 4/20 | 20.0 |
| Mammary gland, carcinoma | 4/22 | 18.2 | 3/20 | 15.0 |
| Mammary gland, other (mixed | 1/22 | 4.5 | 1/20 | 5.0 |
| tumor, pilomatrixoma) | | | | |
| Pancreas, islet cell adenoma | 1/17 | 5.9 | 1/20 | 5.0 |
| Pituitary adenoma, pars distalis | 6/18 | 33.3 | 9/20 | 45.0 |
| Skin, lymphangiosarcoma | 1/18 | 5.6 | 0/19 | 0 |
| Spleen, hemangiosarcoma | 1/18 | 5.6 | 0/20 | 0 |
| Stomach, nonglandular, | 1/19 | 5.3 | 0/20 | 0 |
| squamous carcinoma | | | | |
| Uterus, hemangioma | 1/22 | 4.5 | 0/20 | 0 |
| Uterus, leiomyoma | 2/22 | 9.1 | 0/20 | 0 |

TABLE 3A
Incidence of Non-Neoplastic Microscopic Findings that Show a
Statistically Significant Difference
Between Two Genotype Groups

| | Incidence in | Incidence in | |
|---|---|---|---|
| Phenotype | FVBs | SR2F | p value |
| Kidney, lymphocytic infiltrate | 0/22 (0%) | 7/20 (35%) | 0.003 |
| Lung, lymphocytic infiltrate | 9/17 (53%) | 16/20 (80%) | 0.02 |
| Pancreas, lymphocytic infiltrate | 4/17 (24%) | 12/20 (60%) | 0.04 |
| Chronic cardiomyopathy | 13/19 (68%) | 19/20 (95%) | 0.04 |
| Pancreas, islet cell hyperplasia | 2/17 (12%) | 10/20 (50%) | 0.02 |
| Spinal cord, eosinophilic | 1/17 (6%) | 11/20 (55%) | 0.017 |
| degeneration | | | |
| Thymus, lymphoid hyperplasia, | 2/17 (12%) | 10/19 (53%) | 0.01 |
| medulla | | | |
| Thymus, hyperplasia, medulla | 5/17 (30%) | 0/19 (0%) | 0.01 |

TABLE 3B
Severity summary for all non-neoplastic
lesions showing statistically
significant difference between the genotype groups

| | Incidence in | Incidence in | |
|---|---|---|---|
| Phenotype | FVBs | SR2F | p value |
| Chronic cardiomyopathy: ALL | 13/19 (68%) | 19/20 (95%) | 0.04 |
| SEVERITIES | | | |
| MINIMAL | 3/19 (16%) | 6/20 (30%) | NS |
| MILD | 8/19 (42%) | 11/20 (55%) | NS |
| MODERATE | 2/19 (11%) | 2/20 (10%) | NS |
| Kidney, lymphocytic infiltrate: | 0/22 (0%) | 7/20 (35%) | 0.003 |
| ALL SEVERITIES | | | |
| MINIMAL | 0/22 (0%) | 5/20 (25%) | 0.02 |
| MILD | 0/22 (0%) | 2/20 (10%) | NS |
| Lung, lymphocytic infiltrate: | 9/17 (53%) | 16/20 (80%) | 0.02 |
| ALL SEVERITIES | | | |
| MINIMAL | 5/17 (29%) | 4/20 (20%) | NS |
| MILD | 2/17 (12%) | 10/20 (50%) | 0.02 |
| MODERATE | 1/17 (6%) | 2/20 (10%) | NS |
| MARKED | 1/17 (6%) | 0/20 (0%) | NS |
| Pancreas, islet cell hyperplasia: | 2/17 (12%) | 10/20 (50%) | 0.02 |
| ALL SEVERITIES | | | |
| MINIMAL | 1/17 (6%) | 5/20 (25%) | NS |
| MILD | 1/17 (6%) | 5/20 (25%) | NS |
| Pancreas, lymphocytic infiltrate: | 4/17 (24%) | 12/20 (60%) | 0.04 |
| ALL SEVERITIES | | | |
| MINIMAL | 4/17 (24%) | 11/20 (55%) | NS |
| MILD | 0/17 (0%) | 1/20 (5%) | NS |
| Spinal cord, eosinophilic | 1/17 (6%) | 11/20 (55%) | 0.017 |
| degeneration: ALL | | | |
| SEVERITIES | | | |
| MINIMAL | 1/17 (6%) | 3/20 (15%) | NS |
| MILD | 0/17 (0%) | 3/20 (15%) | NS |
| MODERATE | 0/17 (0%) | 5/20 (25%) | 0.05 |
| Thymus, lymphoid hyperplasia, | 2/17 (12%) | 10/19 (53%) | 0.01 |
| medulla: ALL SEVERITIES | | | |
| MINIMAL | 1/17 (6%) | 1/19 (5%) | NS |
| MILD | 1/17 (6%) | 8/19 (42%) | 0.02 |
| MODERATE | 0/17 (0%) | 1/19 (5%) | NS |
| Thymus, hyperplasia, medulla: | 5/17 (30%) | 0/19 (0%) | 0.01 |
| ALL SEVERITIES | | | |
| MILD | 3/17 (18%) | 0/19 (0%) | NS |
| MODERATE | 2/17 (12%) | 0/19 (0%) | NS |

B. Mammary Gland Phenotyping

Mice were staged in the estrus cycle by examination of vaginal lavages. The #4 (left abdominal) mammary glands were harvested for gross morphological analysis by whole mounting, and the #9 (right abdominal) mammary glands were fixed in neutral buffered formalin and processed for histological analysis. Mammary glands from virgin SR2F transgenic mice were compared with mammary glands from age-matched virgin control mice in the same stage of the estrus cycle. Mammary glands from lactating mice and mice at days 1, 2, 6, and 10 of involution were also compared. Three mice were used for each genotype group.

Since expression of the SR2F was highest in the mammary gland, the mammary gland was examined in greater detail. Whole mount analysis of three 9-week old virgin SR2F+/− mice did not show any significant mammary phenotype when compared with wild-type (data not shown). In particular, there was no evidence for the increased ductal branching or premature lobulo-alveolar development that would have been predicted from the phenotypes of mice overexpressing a dominant negative TGF-β receptor in the mammary stroma or epithelium (Amendt et al., Oncogene, 17:25-34 [1998; Gorska et al., Cell Growth Different., 9:229-238 [1998]; and Joseph et al., Mol. Biol. Cell, 10:1221-1234 [1999]). Mammary epithelium derived from TGF-β3 null mice showed decreased apoptosis in the day 1 involuting gland which suggested that the SR2F mice might show a delay in involution, or a failure to involute correctly (Nguyen et al., Develop., 127:3107-3118 [2000]). However, whole mount and histological analysis of mammary glands at various stages of involution showed no consistent phenotypic differences between SR2F and wild-type mice (data not shown). Furthermore, there were no statistically significant differences in non-neoplastic microscopic findings in the cohort of aged SR2F and FVB/N mice, as shown in Table 4, below, except that the incidence of mammary gland pigmentation was increased in SR2F mice. The denominator in the incidence column of this Table gives the total number of mammary glands examined microscopically from 22 FVB/N mice and 20 SR2F mice.

TABLE 4
Incidence of Non-Neoplastic Microscopic
Findings in the Mammary Gland

| | FVB/N | | | SR2F | | |
|---|---|---|---|---|---|---|
| | | # | | | # | |
| | | glands | | Inci- | glands | Inci- |
| | # Glands | with | dence | # Glands | with | dence |
| | examined | lesion | (%) | examined | lesion | (%) |
| Hyperplasia, | | | | | | |
| physiological | | | | | | |
| without atypia | | | | | | |
| arous mice | 14 | 11 | 79 | 20 | 18 | 90 |
| irgin mice | 40 | 18 | 45 | 32 | 22 | 69 |
| Metaplasia, | 54 | 15 | 28 | 52 | 15 | 29 |
| squamous | | | | | | |
| Dilatation, | 54 | 15 | 28 | 52 | 15 | 29 |
| cystic ductal | | | | | | |
| MIN, low | 54 | 1 | 2 | 52 | 0 | 0 |
| grade | | | | | | |
| Inflammation | 54 | 9 | 17 | 52 | 13 | 25 |
| chronic | | | | | | |
| Lymphocytic | 54 | 1 | 2 | 52 | 2 | 4 |
| infiltrate | | | | | | |
| Mineralization | 54 | 0 | 0 | 52 | 4 | 8 |
| Pigment | 54 | 25 | 46 | 52 | 40 | 77 |

C. Immunophenotyping

Whole blood was collected from 8-10 month old virgin transgenic and wild-type mice by cardiac puncture and complete blood counts (CBCs) were determined by Anilytics (Anilytics, Inc., Gaithersburg, Md.). To determine spleen cell counts, spleens were excised, ruptured and erythrocytes were lysed with ACK lysing buffer (BioWhittaker, Inc., Walkersville, Md.). Spleen cell numbers were then determined by counting using a hemocytometer. For determination of the percentage of CD4+ memory T-cells in the spleen cell population, fluorescent activated cell sorting was done by FACSCalibur using CellQuest software (Becton Dickinson and Co., San Jose, Calif.) Antibodies were used against CD44 and CD62L markers to identify the CD₄₄^{high}, CD62L^{low} sub-population that represents the memory T-cells (Gorelik and Flavell, Immun., 12:171-181 [2000]). For direct staining to determine total leukocyte distribution and to quantitiate the activated and/or memory T cell phenotypes, the following conjugated antibodies were purchased from BD Biosciences, Pharmingen (San Diego, Calif.): anti CD69 FITC, anti-CD25 phycoerythrin (PE), anti-CD62L FITC, anti-CD44 PE, anti-CD4 peridinin chlorophyll (PerCP), allphycocyanin (APC), anti-CD8 PerCP, anti-CD3 APC, anti-B220 APC, anti-NK1.1 PE, anti-CD11b FITC, and anti-Ly-6G (Gr-1) PE. Before staining, Fc receptors were blocked with anti-CD16/32 antibody (BD Biosciences, Pharmingen, San Diego, Calif.).

No change was observed in circulating white cell, red cell and platelet counts in aged SR2F mice, as indicated in Table 5. In this Table, data are presented for seven 8-10 month old wild-type female SR2F+/+ mice, in comparison with seven matched FVB/N controls for circulating blood cell counts, hematocrit parameters and spleen cell counts. In each genotype group, 4 of the mice were virgin and 3 were parous. There were no significant differences between virgin and parous mice (not shown). Not all measurements were made on all mice. In this Table, “WBC” refers to white blood cells, “RBC” refers to red blood cells, “Hb” refers to hemoglobin, “MCV” refers to mean corpuscular volume, “MCH” refers to mean corpuscular hemoglobin, “MCHC” refers to mean corpuscular hemoglobin concentration, “n” refers to the number of mice analyzed in each genotype group and “N/A” means not applicable. Spleen size (not shown) and spleen cellularity (Table 5) were also normal, suggesting there was no splenomegaly as found in the TGF-β1 null mice (Shull et al., supra) nor lymphoid hyperplasia of the type observed in mice with TGF-β response compromised in the T-cell compartment (Gorelik and Flavell, supra).

TABLE 5
Complete blood counts (CBCs) and spleen cell
numbers are unchanged in SR2F mice.

| | n | FVB/N | SR2F | Normal range |
|---|---|---|---|---|
| Age (months) | 7 | 9.5+/−0.4 | 9.0+/−1.0 | N/A |
| Serum SR2F | 3 | 0 | 296+/−63 | N/A |
| (ng/ml) | | | | |
| WBC (ths/ul) | 7 | 7.6+/−1.9 | 8.2+/−3.0 | 2.6-10.7 |
| RBC (mill/ul) | 7 | 7.3+/−1.0 | 7.1+/−0.8 | 6.4-9.4 |
| Platelets | 7 | 1262+/−184 | 1119+/−365 | 592-2972 |
| (thsn/ul) | | | | |
| Hb (g/dl) | 4 | 12.1+/−2.0 | 12.3+/−0.8 | 11.5-16.1 |
| Hematocrit (%) | 4 | 33.8+/−5.6 | 32.6+/−3.6 | 36.1-49.5 |
| MCV (Fl) | 4 | 49.0+/−0.9 | 48.9+/−0.7 | 45.4-60.3 |
| MCH (pg) | 4 | 17.5+/−0.2 | 18.5+/−1.0 | 14.1-19.3 |
| MCHC (%) | 4 | 35.7+/−0.3 | 37.8+/−2.3 | 25.4-34.1 |
| Segmented | 4 | 1.47+/−1.18 | 0.86+/−0.71 | 0.13-2.57 |
| neutrophils | | | | |
| (thsn/ul) | | | | |
| Lymphocytes | 4 | 6.85+/−2.0 | 6.55+/−1.62 | 1.43-9.94 |
| (thsn/ul) | | | | |
| Spleen cells | 4 | 157+/−16 | 156+/−25 | |
| (millions) | | | | |

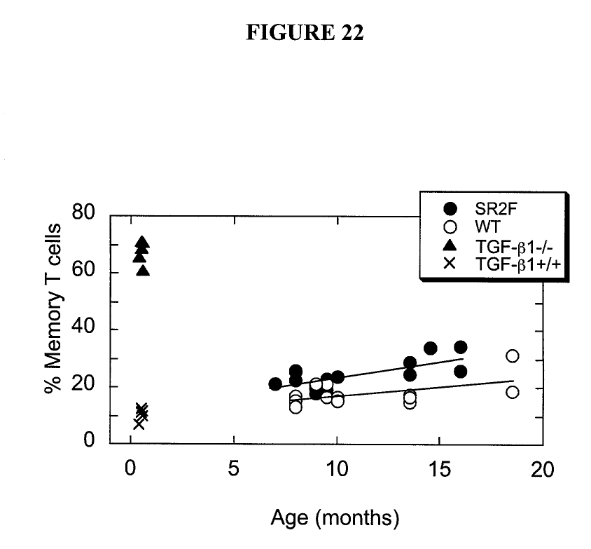

FACS analysis of spleen cells showed that the SR2F mice had the normal proportion of B cells and of CD4 and CD8 single-positive T cells, suggesting that there is no abnormal expansion of lymphocytes in the periphery. More detailed analysis of the splenic T cell population in larger cohorts of mice showed that the expression levels of early activation markers of T cells, such as CD69 and CD25, were the same in MMTV-SR2F mice and wild-type controls. There was a small but significant increase in the fraction of CD4+ T cells with memory phenotype (CD4+CD44^{high}CD62^{low}; see FIG. 22) and in CD8+ memory T cells in older MMTV-SR2F mice when compared with age-matched controls. However, the increased acquisition of memory phenotype was minimal in comparison with that seen in the TGF-β1 null mice (FIG. 22). (The TGF-β1 null mice do not survive beyond about 3 weeks of age).

From the above it is clear that the invention provides improved methods and compositions for the suppression of metastasis by a soluble TGF-β antagonist (SR2F), as well as transgenic non-human animals expressing this antagonist.

All publications and patents mentioned in the above specification are herein incorporated by reference. Various modifications and variations of the described method and system of the invention will be apparent to those skilled in the art without departing from the scope and spirit of the invention. Although the invention has been described in connection with specific preferred embodiments, it should be understood that the invention as claimed should not be unduly limited to such specific embodiments. Indeed, various modifications of the described modes for carrying out the invention which are obvious to those skilled in molecular biology or related fields are intended to be within the scope of the present invention.

We claim:

1. A method for suppressing metastasis, comprising the step of administering a soluble transforming growth factor beta antagonist to a subject having at least one tumor, under conditions such that the normal functions of transforming growth factor beta are not detrimentally affected by said soluble transforming growth factor beta antagonist.

2. The method of claim 1, wherein said soluble transforming growth factor beta antagonist is SR2F.

3. The method of claim 1, wherein said soluble transforming growth factor beta antagonist is an antibody.

4. The method of claim 1, wherein said soluble transforming growth factor beta antagonist is a soluble beta glycan.

5. The method of claim 1, wherein said soluble transforming growth factor beta antagonist is a soluble dominant negative transforming growth factor beta receptor.

6. The method of claim 1, wherein said soluble transforming growth factor beta antagonist is decorin.

7. The method of claim 1, wherein said soluble transforming growth factor beta antagonist is a latency associated peptide.

8. The method of claim 1, wherein said transforming growth factor beta is transforming growth factor beta 1.

9. The method of claim 1, wherein the said transforming growth factor beta is transforming growth factor beta 2.

10. The method of claim 1, wherein the said transforming growth factor beta is transforming growth factor beta 3.

11. The method of claim 1, wherein said subject is a human.

12. The method of claim 1, wherein said tumor is selected from the group consisting of mammary tumors, prostate tumors, colon tumors, gastric tumors, liver tumors, pancreatic tumors, lung tumors, kidney tumors, bladder tumors, nasopharyngeal carcinomas, melanomas, chondrosarcomas and osteosarcomas.

13. The method of claim 1, wherein the tumor is prevented from metastasizing.

14. A transgenic non-human animal comprising a soluble transforming growth factor beta antagonist.

15. The method of claim 14, wherein said soluble transforming growth factor beta antagonist is SR2F.

16. The method of claim 14, wherein said soluble transforming growth factor beta antagonist is an antibody.

17. The method of claim 14, wherein said soluble transforming growth factor beta antagonist is a soluble beta glycan.

18. The method of claim 14, wherein said soluble transforming growth factor beta antagonist is a soluble dominant negative transforming growth factor beta receptor.

19. The method of claim 14, wherein said soluble transforming growth factor beta antagonist is decorin.

20. The method of claim 14, wherein said soluble transforming growth factor beta antagonist is a latency associated peptide.

21. The transgenic non-human animal of claim 14, wherein said soluble transforming growth factor beta antagonist prevents metastasis of tumors in said transgenic animal.

22. The transgenic non-human animal of claim 14, wherein said animal is a rodent.

23. A composition comprising a container with a soluble transforming growth factor beta antagonist and instructions for use for treating cancer in a subject.

24. The method of claim 23, wherein said soluble transforming growth factor beta antagonist is SR2F.

25. The method of claim 23, wherein said soluble transforming growth factor beta antagonist is an antibody.

26. The method of claim 23, wherein said soluble transforming growth factor beta antagonist is a soluble beta glycan.

27. The method of claim 23, wherein said soluble transforming growth factor beta antagonist is a soluble dominant negative transforming growth factor beta receptor.

28. The method of claim 23, wherein said soluble transforming growth factor beta antagonist is decorin.

29. The method of claim 23, wherein said soluble transforming growth factor beta antagonist is a latency associated peptide.

30. A method of treating a subject having cancer comprising the steps of:
a) administering a soluble transforming growth factor beta antagonist
b) and administering a chemotherapeutic agent.

31. The method of claim 30, wherein said soluble transforming growth factor beta antagonist is SR2F.

32. The method of claim 30, wherein said soluble transforming growth factor beta antagonist is an antibody.

33. The method of claim 30, wherein said soluble transforming growth factor beta antagonist is a soluble beta glycan.

34. The method of claim 30, wherein said soluble transforming growth factor beta antagonist is a soluble dominant negative transforming growth factor beta receptor.

35. The method of claim 30, wherein said soluble transforming growth factor beta antagonist is decorin.

36. The method of claim 30, wherein said soluble transforming growth factor beta antagonist is a latency associated peptide.

37. A method for treating a subject having cancer, comprising the step of administering a soluble transforming growth factor beta antagonist to a subject having at least one tumor, under conditions such that the normal functions of transforming growth factor beta are not detrimentally affected by said soluble transforming growth factor beta antagonist.

38. The method of claim 37, wherein said soluble transforming growth factor beta antagonist is SR2F.

39. The method of claim 37, wherein said soluble transforming growth factor beta antagonist is an antibody.

40. The method of claim 37, wherein said soluble transforming growth factor beta antagonist is a soluble beta glycan.

41. The method of claim 37, wherein said soluble transforming growth factor beta antagonist is a soluble dominant negative transforming growth factor beta receptor.

42. The method of claim 37, wherein said soluble transforming growth factor beta antagonist is decorin.

43. The method of claim 37, wherein said soluble transforming growth factor beta antagonist is a latency associated peptide.

44. The method of claim 37, wherein said subject is a human.

45. The method of claim 37, wherein said tumor is selected from the group consisting of mammary tumors, prostate tumors, colon tumors, gastric tumors, liver tumors, pancreatic tumors, lung tumors, kidney tumors, bladder tumors, nasopharyngeal carcinomas, melanomas, chondrosarcomas and osteosarcomas.

46. The method of claim 37, wherein the tumor is prevented from metastasizing.

47. The method of claim 37, wherein said transforming growth factor beta is transforming growth factor beta 1.

48. The method of claim 37, wherein the said transforming growth factor beta is transforming growth factor beta 2.

49. The method of claim 37, wherein the said transforming growth factor beta is transforming growth factor beta 3.

50. A method of treating a subject having cancer comprising the steps of:
a) administering a soluble transforming growth factor beta antagonist
b) and administering an immunotherapeutic agent.

51. The method of claim 50, wherein said soluble transforming growth factor beta antagonist is SR2F.

52. The method of claim 50, wherein said soluble transforming growth factor beta antagonist is an antibody.

53. The method of claim 50, wherein said soluble transforming growth factor beta antagonist is a soluble beta glycan.

54. The method of claim 50, wherein said soluble transforming growth factor beta antagonist is a soluble dominant negative transforming growth factor beta receptor.

55. The method of claim 50, wherein said soluble transforming growth factor beta antagonist is decorin.

56. The method of claim 50, wherein said soluble transforming growth factor beta antagonist is a latency associated peptide.
